(19) United States
(12) Patent Application Publication
(10) Pub. No.: US 2023/0084052 A1
(43) Pub. Date: Mar. 16, 2023

(54) PROXIMITY ASSAY

(71) Applicant: ABSCI CORPORATION, Vancouver, WA (US)

(72) Inventor: Jia Liu, Vancouver, WA (US)

(21) Appl. No.: 17/798,698

(22) PCT Filed: Feb. 11, 2021
(86) PCT No.: PCT/US21/17688

§ 371 Date: Aug. 10, 2022

Related U.S. Application Data
(60) Provisional application No. 62/961,392, filed on Jan. 15, 2020.
(60) Provisional application No. 62/975,152, filed on Feb. 11, 2020.

(51) Int. Cl. G01N 33/542; G01N 33/53; G01N 33/552; G01N 33/563

(52) U.S. Cl. CPC G01N 33/542; G01N 33/5308; G01N 33/552; G01N 33/563; G01N 2470/10

(57) ABSTRACT

The present disclosure provides assay methods for the detection and/or quantification of an analyte in a sample. In some examples, the methods detect and/or quantify an active analyte in a sample.

### CROSS REFERENCE TO RELATED APPLICATIONS

This application is a continuation-in-part of International Application No. PCT/US2021/013734, filed Jan. 15, 2021, and claims the benefit of U.S. Provisional Application No. 62/975,152, filed Feb. 11, 2020, both of which are incorporated herein by reference in their entirety.

### FIELD

The present invention is in the general technical fields of molecular biology and biotechnological manufacturing. More particularly, the present invention is in the technical field of biotechnological assays for the quantification of analytes in a sample.

### BACKGROUND

Biotechnological manufacturing (or “biomanufacturing”) of biological materials, such as therapeutic proteins or immunogenic compounds, requires quantification of these materials even in the presence of a complex mixture of other cellular components. Many assays that detect the presence of an analyte in a biological composition or sample require expensive reagents and/or are not suitable for rapid, high-throughput screening of host cell lines or other biomanufacturing components or processes.

In addition, many biological materials that are produced through biomanufacturing are complex, requiring the correct association of multiple components and/or the proper folding of the component(s) to achieve a functional structure. Many assays simply detect the presence of an analyte and are not capable of distinguishing between correctly and incorrectly folded analyte, or between active and inactive analyte.

Improved assays capable of high-throughput detection and quantification of analytes, and specifically of active analytes, are clearly needed.

### SUMMARY

The present disclosure provides assay methods for the detection and quantification of analyte, such as active analyte in a sample.

In some embodiments, the methods include: selecting a population of host cells having a genetic diversity, the genetic diversity including a plurality of genetic variants, wherein at least some of the population of host cells include a polynucleotide encoding the analyte; combining a sample including a population of host cells including a single genetic variant from the selected population of host cells and wherein the population of host cells expresses the analyte, a first complex including a signal donor and a first analyte-associating moiety, and a second complex including an activatable compound and a second analyte-associating moiety; wherein the sample comprises active analyte that is in the active form of the analyte and inactive analyte that is not in the active form of the analyte; and wherein the active analyte associates with both the first complex and the second complex, and the inactive analyte does not associate with both the first complex and the second complex; initiating the transfer of a signal from the signal donor in the first complex, wherein the signal is received by the activatable compound in the second complex that is associated with an active analyte associated with both the first complex and the second complex; and detecting the output from the activatable compound.

In other embodiments, the methods include: selecting a population of host cells having a genetic diversity, the genetic diversity including a plurality of genetic variants, wherein at least some of the population of host cells include a polynucleotide encoding the analyte; combining a sample including a population of host cells including a single genetic variant from the selected population of host cells and wherein the population of host cells expresses the analyte with a first complex including a signal donor and a first analyte-associating moiety, and a second complex including an activatable compound and a second analyte-associating moiety; initiating the transfer of a signal from the signal donor to the activatable compound, wherein the signal causes the activatable compound to produce detectable output; and detecting the output from the activatable compound. In some examples, the sample includes active analyte that is in the active form of the analyte and inactive analyte that is not in the active form of the analyte, wherein the active analyte associates with both the first complex and the second complex, and the inactive analyte does not associate with both the first complex and the second complex.

In additional embodiments, the method further includes: contacting the sample with a third complex comprising a second activatable compound and a third analyte-associating moiety; wherein the transfer of the signal from the signal donor to the second activatable compound causes the second activatable compound to produce detectable output; and detecting the output from the second activatable compound.

In other embodiments, the methods include: selecting a population of host cells having a genetic diversity, the genetic diversity including a plurality of genetic variants, wherein at least some of the population of host cells include a polynucleotide encoding the analyte; providing a covalently labeled analyte that includes the analyte connected through a covalent bond to a first detection reagent selected from the group consisting of a signal donor and an activatable compound; contacting a sample including a population of host cells including a single genetic variant from the selected population of host cells and wherein the population of host cells expresses the analyte with the covalently labeled analyte and with an analyte-associating moiety connected to a second detection reagent selected from the group consisting of a signal donor and an activatable compound, wherein the first detection reagent is not the second detection reagent; initiating the transfer of a signal from the signal donor to the activatable compound, wherein the signal causes the activatable compound to produce detectable output; and detecting the output from the activatable compound. In some examples, the sample includes active analyte that is in the active form of the analyte and inactive analyte that is not in the active form of the analyte, wherein the active analyte associates with both the first complex and the second complex, and the inactive analyte does not associate with both the first complex and the second complex.

In some embodiments of the disclosed methods, selecting the population of host cells having genetic diversity and encoding the analyte includes: culturing a population of host cells, whereby the analyte is expressed by a subpopulation of the host cells of the population, the subpopulation thereby including expressing host cells; labeling at least some of the expressing host cells of the subpopulation, wherein the labeling includes associating the gene product of interest with a detectable moiety, thereby producing labeled expressing host cells; and selecting a subset of labeled expressing host cells, wherein the selecting includes detecting the detectable moiety by a cell-sorting apparatus.

In some embodiments, the analyte includes a polypeptide, a protein, a glycoprotein, a phosphoprotein, a proteolipid, an antibody, a polypeptide comprising one or more domains or fragments of any of the preceding, and a nucleic acid. In some examples, the analyte is a homomultimer and/or the analyte has properly formed disulfide bonds. In particular examples, the analyte is a homomultimer and the analyte-associating moiety of the first complex is the same as the analyte-associating moiety of the second complex.

In some embodiments, the method further includes contacting an assay component with a first antibody that specifically binds the assay component, where the assay component is selected from the group consisting of the analyte and the analyte-associating moiety. In additional embodiments, the method further includes contacting the first antibody with a second antibody that specifically binds the first antibody.

In additional embodiments, the analyte-associating moiety is a multimer that can interact with the analyte at multiple sites on the analyte.

In some embodiments, the analyte-associating moiety is attached to a detection reagent selected from the group consisting of a signal donor and an activatable complex. In examples, the analyte-associating moiety is attached to the detection reagent through a connector, such as a polypeptide linker and/or a binding pair.

In further embodiments, the signal donor is activated by an enzyme and/or the signal donor is activated by irradiation. In some examples, the signal donor produces a fluorescence resonance transfer signal, and/or the signal donor produces a chemical signal. In one example, the chemical signal is a reactive oxygen species. In other embodiments, the signal donor is a sensitizer, such as a haloperoxidase or a photosensitizer. In further embodiments, the activatable compound is a photoactivable compound. In some examples, photoactivable compound emits light by fluorescence. In other examples, the photoactivable compound undergoes a chemical reaction with singlet oxygen.

In some embodiments, the methods further include measuring the optical density of the sample. In other embodiments, the methods further include: adding a compound that interacts with nucleic acid to the sample, irradiating the sample to excite the compound that interacts with nucleic acid, and measuring the light emitted by the excited compound.

In additional embodiments, at least one assay component selected from the group consisting of the analyte, an analyte-associating moiety, the signal donor, and the activatable compound is attached to a solid support. In some examples, the solid support is in the form of a bead.

In some embodiments, a further assay is performed on the sample, and the assay is selected from the group consisting of bio-layer interferometry, DNA sequencing, enzyme-linked immunosorbent assay (ELISA), immunofluorescence staining, affinity chromatography, high-performance liquid chromatography (HP-LC), liquid chromatography mass spectrometry (LC-MS), size-exclusion chromatography, solid-phase extraction mass spectrometry (SPE-MS), and surface plasmon resonance.

The foregoing and other features of the disclosure will become more apparent from the following detailed description, which proceeds with reference to the accompanying figures.

### BRIEF DESCRIPTION OF THE DRAWINGS

FIG. 1 is a schematic illustration showing an analyte being contacted by two analyte-associating moieties, ‘A’ and ‘B,’ which are connected to a signal donor and to an activatable compound, respectively. Each analyte-associating moiety can optionally be connected to the signal donor or to the activatable compound through a ‘connector’ molecule or complex of molecules. In certain embodiments, an initiation event is required for the signal donor to produce signal. The signal donor produces a signal that is capable of activating the activatable compound; if the activatable compound is in close enough proximity to the signal donor to receive the signal, the activatable compound is activated to generate an output that can be detected.

FIG. 2 is a schematic illustration showing the same configuration of assay components as in FIG. 1, except in this case the analyte is an antibody. For homodimeric antibodies where the antigen-binding domains recognize the same antigen, the analyte-associating moieties ‘A’ and ‘B’ can both be that antigen. For bispecific antibodies, the analyte-associating moieties ‘A’ and ‘B’ can be different antigens that are recognized by the antibody.

FIG. 3 is a schematic illustration of an alternative assay method of the invention. In cases where only one analyte-associating moiety is used to contact the analyte, a competitive binding assay can be used to detect and quantify analyte in a sample. The analyte is connected to the activatable compound (as shown), or alternatively to the signal donor, and the analyte-associating moiety is connected to the signal donor (as shown) or to the activatable compound, respectively. The analyte is connected to the activatable compound (or to the signal donor) through a covalent connection, represented by a shaded bar, which in the illustrated embodiment covalently attaches an optional connector to the analyte, to form a covalently labeled analyte. When the covalently labeled analyte and the analyte-associating moiety are allowed to associate, a detectable output is generated as described in FIGS. 1 and 2. When unlabeled analyte in a sample is assayed by addition of the covalently labeled analyte and other detection reagents, the covalent labeling of the covalently labeled analyte limits the competition between the covalently labeled analyte and the unlabeled analyte to competition for interaction with the analyte-associating moiety, as represented by the large shaded arrow. The presence of unlabeled analyte in the sample is detected by the reduction in the level of the detectable output, resulting from the competitive association of the unlabeled analyte with the analyte-associating moiety, disassociating the covalently labeled analyte, and disrupting the reception of the signal by the activatable compound.

FIG. 4A is a schematic illustration of a particular embodiment of the methods, in which antibodies are used to amplify the detectable output of the assay. The small circles labeled ‘A’ and ‘B’ are analyte-associating moieties, each attached by an optional connector (thick shaded line) to a signal donor (large shaded circle) for ‘A’ analyte-associating moieties, or to an activatable compound (large open circle) for ‘B’ analyte-associating moieties. This is a simplified version of the arrangement of analyte-associating moieties, optional connectors, signal donors, and activatable compounds shown in FIGS. 1 and 2; this version does not depict any of the arrows representing the signals which are transmitted from each of the signal donors to the activatable compounds. The antibodies labeled ‘C’ represent the analyte. The antibodies labeled ‘D’ are antibodies specific for antibody ‘C,’ and the antibody labeled ‘E’ is an antibody specific for the ‘D’ antibodies. The aggregation of analytes by these antibodies causes additional signal donors and activatable compounds to be brought into proximity with each other through their association with the analyte, allowing multiple signal donors to transmit signal which can be received by multiple activatable compounds, resulting in a potentially exponential increase in the detectable output (outward-directed lightning bolts). This is particularly useful for homodimeric analytes that happen to interact with analyte-associating moieties attached only to signal donors, or only to activatable compounds as shown in the right-most portion of this figure; in that situation, the aggregation of analytes will allow for the reception by activatable compounds of signal from proximal signal donor(s).

FIG. 4B is a schematic illustration of a particular embodiment of the methods, in which a signal boost is obtained by multimerizing both the analyte (for example, as illustrated in FIG. 4A) and the analyte-associating moiety (antigen in this case). This is due to the avidity principle: the apparent dissociation constant is lowered because the apparent off-rate is no longer based on a single analyte-analyte-associating moiety interaction. FIG. 4 shows each signal donor and activatable compound as being linked to multiple analyte-associating moieties (‘A’ and ‘B,’ respectively). The apparent off-rate for a given analyte-associating moiety in this case is now the product of multiple off rates. The signal boost comes from the increased probability of a signal donor in proximity to an activatable compound. In actual practice, both the signal donor and activatable compound are coated with many molecules of the analyte-associating moiety.

FIG. 5 a schematic illustration of an embodiment showing an analyte being contacted by two analyte-associating moieties, ‘A’ and ‘B,’ which are connected to a signal donor and to a first activatable compound (activatable compound 1), respectively. The analyte is also contacted by a third analyte-associating moiety, V,′ which is connected to a second activatable compound (activatable compound 2), which is different from activatable compound 1, but is also compatible with the signal donor. Each analyte-associating moiety can optionally be connected to the signal donor or to the activatable compound through a ‘connector’ molecule or complex of molecules. In certain embodiments, an initiation event is required for the signal donor to produce signal. The signal donor produces a signal that is capable of activating each of the activatable compounds; if the activatable compound is in close enough proximity to the signal donor to receive the signal, the activatable compound is activated to generate output that can be detected. If both activatable compounds 1 and 2 are in close enough proximity to the signal donor, two distinct outputs are generated.

FIG. 6 is a schematic illustration of an exemplary workflow showing a population of host cells with high genetic diversity that is screened with an activity specific cell enrichment assay, generating a population of host cells with lower genetic diversity. The population of host cells with lower genetic diversity is enriched for an analyte of interest or activity of interest. The population of host cells with lower genetic diversity is selected and screened with a proximity assay disclosed herein, resulting in a population of host cells with low genetic diversity, which are further enriched for the analyte or activity of interest. Analytics and fermentation optimization are performed and a final “optimized” strain is identified.

FIG. 7 is a graph showing the correlation between the quantification of active trastuzumab by a proximity assay of the disclosure (x-axis), and the quantification of active trastuzumab by size-exclusion chromatography (SEC) following purification by protein A (ProA) (‘ProA/SEC’, y-axis). Trastuzumab was produced by host cells in a series of experiments using varying expression vectors, cell growth conditions, and induction conditions. For each of these experiments, the proximity assay and ProA SEC were used on the same samples to determine the amount, in grams per liter of fermentation culture (g/L), of trastuzumab produced by the host cells. The points on the graph marked by triangles indicate three outlier points out of a total of 56 data points. When the outlier points are disregarded and a line is plotted on the basis of the remaining points, the coefficient of correlation (r) between the quantification methods is 0.939457.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

| SEQ | | | | Description; |
|---|---|---|---|---|
| ID NO: | Length: | Type: | Organism: | ‘Other Information’ |
| 1 | 451 | PRT | Artificial | Trastuzumab heavy chain |
| | | | Sequence | |
| 2 | 215 | PRT | Artificial | Trastuzumab light chain |
| | | | Sequence | |
| 3 | 15 | PRT | Artificial | AviTag ™ peptide for covalent |
| | | | Sequence | attachment of biotin |

### DETAILED DESCRIPTION

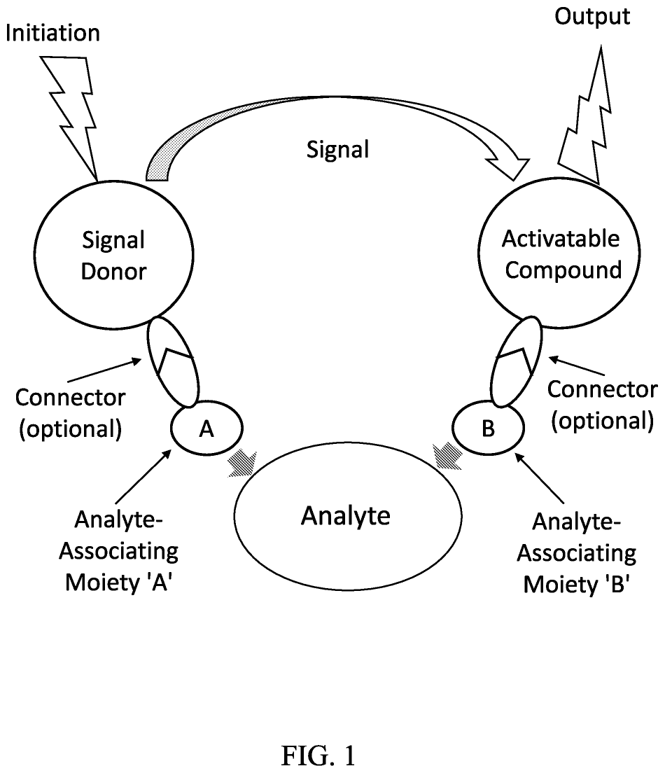

The problem of detecting and quantifying an analyte in a biological sample or mixture is addressed by providing the assay methods described herein. The assay methods comprise the use of assay components that bind to the analyte in a manner that allows for detection of the analyte, as diagrammed in FIGS. 1-5, and particularly for detection of active or intact (e.g., fully-assembled) analyte. In certain embodiments, the analyte can be quantified by comparison to compositions comprising known amounts of the analyte. The assay components, which are also referred to as detection reagents, include the analyte, the analyte-associating moiety or moieties, the signal donor, the activatable compound(s), any connectors that attach the analyte-associating moiety or moieties to the signal donor or the activatable compound(s), and any additional components such as DNA dyes that are added to enhance the assay's capabilities. “Attachment” means a non-transient interaction between molecules, which can be through a covalent bond or through a non-covalent association.

The analyte and the assay components are described in sections I-IV below, and further aspects of the assay method are described in sections V and VI below.

### I. Analytes

The assay methods of the invention are suitable for the detection of any type of molecule or complex of molecules, that is capable of being bound by one or more analyte-associating moieties. Examples of analytes include but are not limited to peptides, polypeptides, proteins, glycoproteins, nucleic acids, phosphoproteins, proteolipids, adhesins, antibodies, antigens, cytokines, enzymes, growth factors, ligands, receptors, structural proteins, transcription factors, transporter proteins, toxins such as peptides (peptide toxins) and proteins that act as toxins (protein toxins), and domains and fragments thereof.

An active analyte is capable of demonstrating one or more activities, such as an enzymatic activity, and/or a binding activity (binding a binding partner), and/or an activity involving a particular conformational change. The active form of the analyte is achieved through the correct association of its components, if it is not a unitary molecule, and the proper folding of its component(s). Detection of an analyte in its properly folded form is an indication that the analyte is active. Determining whether an analyte is properly folded can involve the use of antibodies that specifically bind to the properly folded and active analyte: binding of such antibodies to the analyte is an indication that the analyte is active. For analytes that comprise one or more disulfide bonds, another indication of the activity of the analyte is the presence of the proper number of disulfide bonds in the correct locations, which can be ascertained as described in Example 3. For analytes that are enzymes, the ability to bind the enzymatic substrate or an analog thereof is an indication that the analyte has enzymatic activity. For analytes that are antibodies or antibody fragments, the ability to bind antigen demonstrates that the analyte has antigen-binding activity.

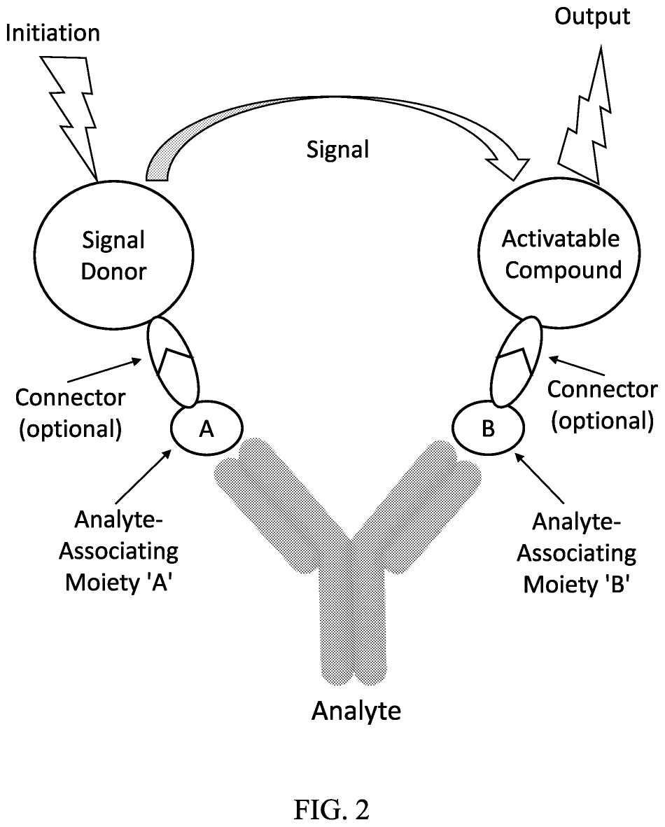

Certain advantageous embodiments of the invention involve methods to detect and quantify analytes that are homomultimers, such as homodimers, homotrimers, homotetramers, and higher-order homomultimers. For analytes that are homomultimers, such as homodimeric antibodies that bind the same antigen at each of their antigen-binding sites, only one type of analyte-associating moiety needs to be used to bring the signal donor into proximity with the activatable compound (see FIG. 2).

### II. Analyte-Associating Moieties

In certain methods disclosed herein, the analyte is contacted with two or more analyte-associating moieties, each of which associates with the analyte at a functionally distinct site. When the analyte is a homomultimer as described above, with multiple identical potential binding sites for an analyte-associating moiety, preferably only one type of analyte-associating moiety is used to contact the analyte.

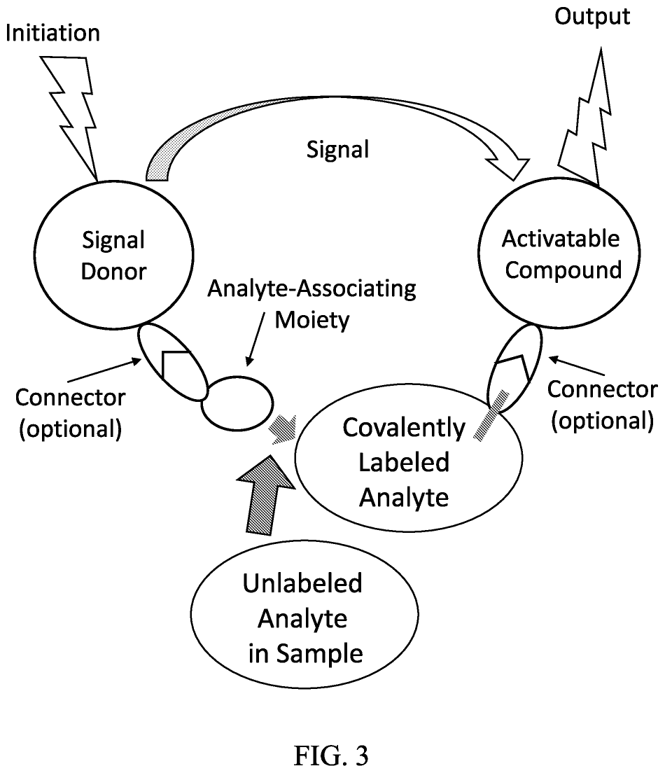

In instances where the analyte is not a homomultimer and only one analyte-associating moiety is available for a particular analyte, the presence of unlabeled analyte in a sample can be detected by allowing unlabeled analyte to compete with covalently labeled analyte and measuring the resulting decrease in signal from the assay components, as shown schematically in FIG. 3.

Preferably, each analyte-associating moiety that is used in the methods provided interacts with the analyte in a highly specific manner. In certain methods, it is also preferable that at least one of the analyte-associating moieties used to contact the analyte is specific for the properly folded, and thus presumptively active, form of the analyte. Inactive analytes can have structural differences from the active form of the analyte, and these structural differences can prevent association between inactive analyte and one or more analyte-associating moieties, so that signal is not received by the activatable compound and detectable output is not generated. Examples of analyte-associating moieties that are specific for an active form of the analyte are antigens for antibody and antibody-fragment analytes, substrates and substrate analogs for enzyme analytes, ligands and ligand analogs for receptors, and antibodies that are specific for the properly folded form of any analyte. For example, for analytes that comprise antigen-binding sites, the binding of antigen (an analyte-associating moiety) to the analyte requires an antigen-binding site formed by the proper interaction of regions within the analyte (e.g., heavy chain variable regions and light chain variable regions). Successful interaction between the analyte-associating moiety (antigen) and the analyte (comprising an antigen-binding site) can indicate that the analyte is active. Further, it is preferable that the analyte-associating moiety interacts with the analyte with high affinity and/or avidity, particularly in assay methods where the analyte is to be detected in a complex mixture such as a cell lysate. For example, the interaction between the analyte-associating moiety and the analyte should withstand competition from other molecules in the complex mixture.

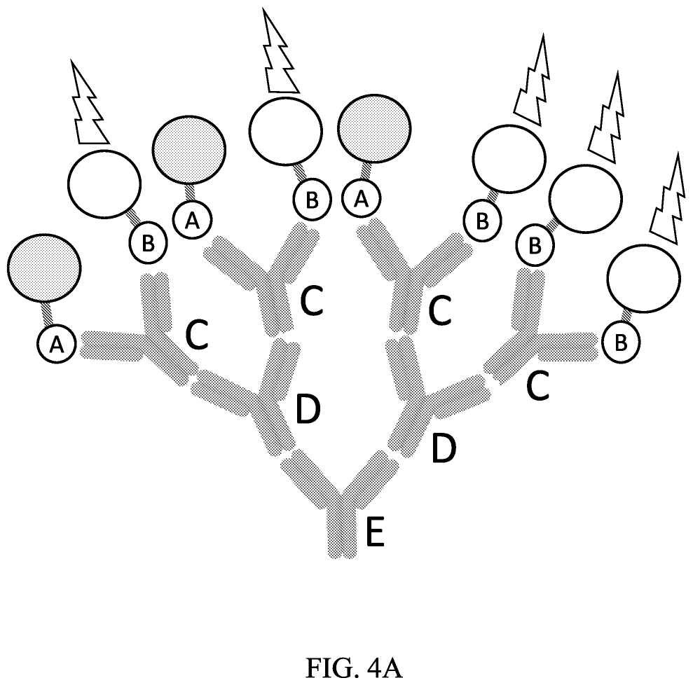

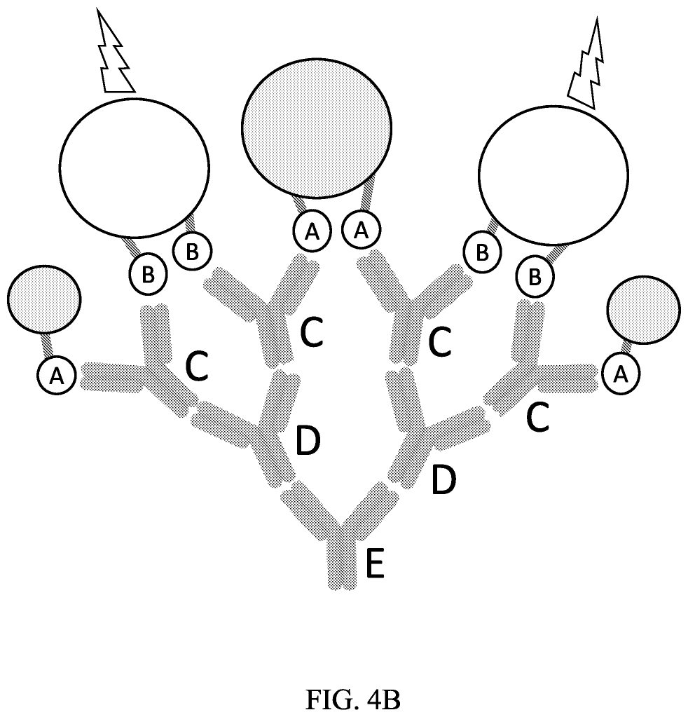

To increase the detectable output generated by weakly binding analyte-associating moieties, these moieties can be attached to more than one connector molecule such as biotin, optionally through linker molecules such as polypeptides, allowing more than one signal donor or activatable compound to be attached to the weakly binding analyte-associating moiety, which amplifies the detectable output created by interactions with the analyte that do occur. Another example of a method of amplifying the detectable output is shown in FIG. 4A, in which antibodies are used to aggregate an analyte. This example has an antibody (labeled ‘C’ in FIG. 4A) as the analyte, but it is applicable to any analyte or analyte-associating moiety that can be bound by an antibody without substantially interfering with the interaction(s) between the analyte and the analyte-associating moiety or moieties. Antibodies specific for antibodies raised in a different host species (‘anti-species antibodies’) are particularly useful in this embodiment of the invention. If, for example, antibody ‘C’ is a human or humanized antibody, then antibody ‘D’ could be a goat anti-human antibody, and antibody ‘E’ could be a rabbit anti-goat antibody. This use of anti-species antibodies to aggregate the analyte can be continued for further rounds of aggregation as long as suitable antibodies can be found, for example, until a sufficient level of signal is obtained. However, any agent that can multimerize the analyte or the analyte-associating moiety can create the avidity effect. For example, in FIGS. 4A and 4B, antibody ‘D’ and antibody ‘E’ could be replaced by non-antibody reagent(s) with appropriate specific multimerization properties.

For an analyte-associating moiety that interacts with the analyte at low affinity, it is possible to increase the overall avidity of the detection reaction. Avidity is a function of the interaction between two multivalent binding partners, in which the ability to bind at multiple binding sites increases the overall strength of the binding interaction. If one binding partner is not multivalent but can become effectively multivalent through multimerization, that can lead to an interaction between the binding partners with an avidity that is significantly higher than the affinity to the monomeric form of that binding partner. As an example, an analyte-associating moiety interacts weakly with an analyte that is sufficiently multivalent to have additional interaction sites beyond those required for interacting with both a signal-donor-attached analyte-associating moiety and an activatable-compound-attached analyte-associating moiety. In this example, multimerization of the weakly interacting analyte-associating moiety, optionally including linker molecules between subunits of the multimer, can allow the analyte-associating moiety to interact at multiple sites on the analyte for a stronger overall interaction, such as is shown in FIG. 4B. This method of increasing the binding strength of the analyte-associating moiety through multimerization can be combined with a method for amplifying the detectable output of the assay, such as is shown in FIG. 4A.

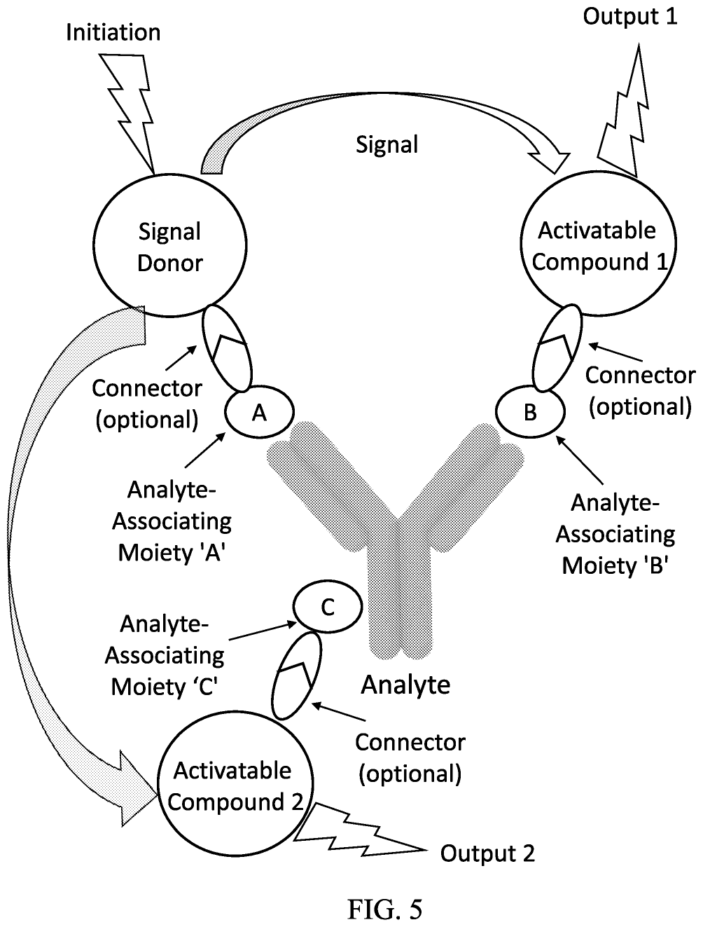

In additional embodiments, the methods include a second activatable compound and at least one additional analyte-associating moiety (such as a third analyte-associating moiety) that is different from the analyte-associating moieties ‘A’ and ‘B’ described in the embodiments shown in FIGS. 1, 2, and 4. With addition of the third analyte-associating moiety (analyte-associating moiety ‘C’ in FIG. 5), it is possible to detect presence of a further binding site on the analyte. Thus, in some examples, this method can be used to detect an intact analyte, such as a fully assembled multimeric analyte. The activatable compounds associated with the analyte-associating moieties are activated by the same signal donor, but have distinguishable outputs. Thus, presence of both of the binding sites for analyte-associating moieties ‘B’ and ‘C’ in close proximity to the binding site for analyte-associating moiety ‘A’ can be identified by detecting presence of outputs from both of the activatable compounds associated with analyte-associating moieties ‘B’ and ‘C.’ In one non-limiting example, the analyte is an antibody, and in this case, analyte-associating moieties ‘A’ and ‘B’ bind to the antigen-binding sites and analyte-associating moiety ‘C’ binds to the Fc domain.

### III. Connectors

In the assay methods provided herein, each analyte-associating moiety is connected to a signal donor or to an activatable compound so that the association of the analyte-associating moieties with the analyte will bring the signal donor and activatable compound into proximity, resulting in a detectable output. The connection between the analyte-associating moiety and the signal donor or the activatable compound can be a direct covalent connection. In some cases the analyte-associating moiety—antigen, ligand, substrate, substrate analog, antibody, etc.—is commercially available as a conjugate with a signal donor or an activatable compound.

It is often necessary or desirable to connect the analyte-associating moiety to the signal donor or to the activatable compound through some type of ‘connector,’ for example, as indicated schematically in FIGS. 1 and 2. It is preferable that the size of each connector molecule is compatible with the signal donor and the activatable compound being brought into proximity by the analyte. As an example of a type of connector, the analyte-associating moiety and the signal donor or the activatable compound can be connected covalently through a linker molecule. An example of a linker molecule is a polypeptide that is used to connect two other polypeptides.

As another example, the analyte-associating moiety can be connected to the signal donor or the activatable compound by covalent linkage of the analyte-associating moiety to one member of a binding pair, and covalent linkage of the signal donor and the activatable compound to the other member of the binding pair. Preferably, binding pairs interact specifically with each other, and preferably with high affinity and/or avidity. Examples of binding pairs include biotin and streptavidin or avidin, poly-histidine and nickel ion (particularly Ni²⁺) or cobalt ion (particularly Co²⁺), an antigen-binding domain and its antigen, and a ligand and its receptor domain. Another example of a binding pair is the SpyTag-SpyCatcher pair. SpyTag is a peptide of 13 amino acids that is bound by the 12.3 kDa SpyCatcher protein, resulting in a covalent intermolecular isopeptide bond.

The use of a binding pair to connect the analyte-associating moiety to the signal donor or to the activatable compound is convenient, in that it allows a signal donor or an activatable compound covalently linked to one member of a binding pair to be connected to any analyte-associating moiety covalently linked to the other member of the binding pair. In some cases the analyte-associating moiety is commercially available as a conjugate with a member of a binding pair, such as biotin. In situations where no such conjugates are commercially available, a member of a binding pair such as biotin can be conjugated to the analyte-associating moiety; biotinylation kits such as the EZ-Link™ Sulfo-NHS-Biotinylation Kit and protocols are available from ThermoFisher Scientific (Waltham, Mass.). Alternatively, the analyte-associating moiety can be produced in a manner that includes a member of a binding pair in its structure, for example by including the AviTag™ peptide (SEQ ID NO:3; Avidity, Aurora, Colo.), which is a target for biotinylation by biotin ligase, or a poly-histidine sequence, or the SpyTag amino acid sequence, in the polypeptide sequence of an analyte-associating moiety when it is expressed in a host cell.

### IV. Signal Donors and Activatable Compounds

A. Signal Donors. A signal donor is a compound capable of providing a signal to an activatable compound. Signal donors include compounds that produce a chemical signal, such as a reactive oxygen species, or a fluorescence resonance transfer signal, or any other signal that can be received by an activatable compound to trigger activation of the activatable compound. In certain embodiments, an initiation event is required for the signal donor to produce a signal, for example, as illustrated in FIGS. 1-3. Other embodiments include signal donors that can generate a signal without an initiation event, for example, the spontaneous emission of particles and/or photons from radioactive material. An initiation event can be an enzymatic activation of the signal donor, as in the activation of luciferin by luciferase, or irradiation of the signal donor at certain wavelengths that causes the signal donor to produce a signal. The initiation event is referred to as ‘excitation’ when the initiation event causes the signal donor to transition to an energetically more excited state.

Certain signal donors that can be used in the proximity assay are sensitizers, molecules which can generate a reactive oxygen species, preferably singlet oxygen. Examples of sensitizers include enzymes and photosensitizers. Enzymes which function as sensitizers include haloperoxidases which form singlet oxygen by catalyzing the reaction of a halide-compound, such as a sodium halide, with hydrogen peroxide. Photosensitizers are molecules which can be excited to a metastable state, usually a triplet state, which in the proximity of molecular oxygen can directly or indirectly transfer its energy to the oxygen molecule, with concomitant excitation of the oxygen to a highly reactive excited state often referred to as singlet oxygen. Singlet oxygen, also called dioxygen (singlet) and dioxidene, is a gaseous inorganic chemical with the formula O=O; within its 4-microsecond half-life, singlet oxygen can diffuse approximately 200 nm in solution, acting as a chemical signal produced by the sensitizer signal donor.

Photosensitizers will usually be excited by the absorption of light or by an energy transfer from an excited state of a suitable donor, but may also be excited by chemiexcitation, electrochemical activation, or by other means. Often excitation of the photosensitizer will be caused by irradiation with light from an external source. Suitable photosensitizers will usually have an absorption maximum in the wavelength range of 250-1100 nm, preferably 300-1000 nm, and more preferably 450-950 nm, with an extinction coefficient at its absorbance maximum in the range of 500 M-1cm-1-100,000 M-1cm-1, or greater than 500 M-1cm-1, preferably at least 5,000 M-1cm-1, and more preferably at least 50,000 M-1cm-1. The lifetime of the excited state, usually a triplet state, produced following absorption of light by the photosensitizer will usually be at least 100 nsec, and preferably at least 1 microsecond in the absence of oxygen. In general, the lifetime must be sufficiently long to permit the energy transfer to oxygen, which will normally be present at concentrations in the range of 0.01 mM to 10 mM (depending on the medium). The excited state of the photosensitizer will usually have a different spin quantum number (S) than its ground state and will usually be in a triplet (S=1) state when, as is usually the case, the ground state is a singlet (S=0). Preferably, excitation of a photosensitizer will produce the long-lived state (usually triplet) with an efficiency of at least 10%, desirably at least 40%, and preferably greater than 80%. The photosensitizer will usually be at most weakly fluorescent under the assay conditions (quantum yield usually less than 0.5, preferably less than 0.1).

Photosensitizers are relatively photostable and will not react efficiently with the singlet molecular oxygen so generated. Several structural features are present in most of the useful photosensitizers, which tend to have at least one and frequently three or more conjugated double or triple bonds held in a rigid, frequently aromatic structure. They will frequently contain at least one group such as a carbonyl or imine group or a heavy atom selected from rows 3-6 of the periodic table, especially iodine or bromine, or they will frequently have polyaromatic structures. Typical photosensitizers include ketones such as benzophenone and 9-thioxanthone; xanthenes such as eosin and rose bengal; polyaromatic compounds such as buckminsterfullerene and 9,10-dibromoanthracene; porphyrins including metallo-porphyrins such as hematoporphyrin and chlorophylis; oxazines; squarate dyes; cyanines such as phthalocyanines, naphthalocyanines, and merocyanines; thiazines such as methylene blue, etc., and derivatives of these compounds substituted by an organic group for rendering such compounds more lipophilic or more hydrophilic and/or as groups for attachment, for example, to a polynucleotide. Examples of other photosensitizers that may be utilized in the present invention are those that have the above properties and are enumerated in N. J. Turro, “Molecular Photochemistry,” page 132, W. A. Benjamin Inc., N.Y. 1965.

B. Signal. The signal produced by the signal donor preferably is received directly by the activatable compound, but the signal can also be transmitted to the activatable compound through an intermediate molecule or compound. However, the reception of the signal by the activatable compound should be a reliable indicator of the physical proximity of the signal donor and the activatable compound, so the use of an intermediate molecule or compound to transmit the signal should not extend the scope of the signal to activatable compounds that are not associated with the same analyte as the signal donor that produced the signal. The signal produced by a signal donor should also be likely to reach only the activatable compound associated with the same analyte in the time frame in which the initiation of signal production and the detection of the output of the activatable compound occurs. If it is desirable to use a signal donor that produces a more widely dispersed signal, it is possible to use an assay medium, or to place or embed one or more assay components on a solid medium or in a colloid such as a gel, where the medium acts as a partial ‘sink’ of the signal, reducing its effective scope. The reception of the signal by the activatable compound is preferably also not sensitive to the relative orientation of the signal donor and activatable compound.

C. Activatable Compounds. An activatable compound refers to a substance that undergoes a chemical, fluorescence, or other reaction upon direct or sensitized excitation by signal from the signal donor. The term “activatable” includes photoactivatable, photochemically activatable, and chemically activatable.

A photoactivable compound is a molecule that, following excitation, emits light, for example by phosphorescence or more preferably by fluorescence. Fluorescence is emission of light following excitation by any suitable means including absorption of light, absorption of x-rays, electrochemical excitation, and chemical excitation. Preferably the emission quantum yield will be high, such as within the range of 0.05 to 1.0, and usually at least 0.1, preferably at least 0.4, more preferably greater than 0.7, and the extinction coefficient of the absorption maximum will usually be greater than 5000 M⁻¹ cm⁻¹. Photoactivable compounds are typically fluorescent compounds, such as fluorescent brighteners, which typically absorb light between 300 and 600 nanometers and emit between 400 and 800 nanometers; xanthenes such as rhodamine and fluorescein; bimanes; coumarins such as umbelliferone; aromatic amines such as dansyl; squarate dyes; benzofurans; cyanines including merocyanines and phthalocyanines; rare earth chelates; porphyrins; polyaromatic compounds such as pyrene, anthracene, and acenaphthene; and chromenes.

An example of an activatable compound is a singlet-oxygen activatable chemiluminescent compound (SACC), which is a photoactivatable substance that undergoes a chemical reaction with singlet oxygen, to form a metastable reaction product that is capable of decomposition with the simultaneous or subsequent emission of light, usually within the wavelength range of 250 to 1200 nm, and preferably within the range of 600 to 800 nm. Activatable compounds of this type can often be electron-rich organic molecules such as enol ethers, enamines, 9-alkylidene-N-alkylacridans, arylvinylethers, dioxenes, arylimidazoles, 9-alkylidene-xanthanes, and lucigenin. These compounds can react with singlet oxygen to form dioxetane or dioxetanone intermediates, which undergo rearrangement resulting in the release of a CO2 molecule and light. Other examples of activatable compounds include luminol and other phthalhydrazides and chemiluminescent compounds that are protected from undergoing a chemiluminescent reaction by virtue of their being protected by a photochemically labile protecting group, such compounds including, for example, firefly luciferin, aquaphorin, luminol, etc.

In particular embodiments the activatable compound emits a detectable signal upon activation, such as light that can be detected by an appropriate detector. In certain embodiments, the activatable compound will preferably emit at a wavelength above 300 nanometers, preferably above 500 nanometers, and more preferably above 550 nm. Compounds that absorb and emit light at wavelengths beyond the region where the contents of the analyte sample contribute significantly to light absorption are of particular use. The absorbance of serum drops off rapidly above 500 nm and becomes insignificant above 600 nm; therefore, activatable compounds that emit light above 600 nm are of particular interest. However, activatable compounds that absorb at shorter wavelengths are useful when interference absorbance of the sample is absent.

D. Measuring the Output of the Activatable Compound. Any suitable detection method can be used to measure the output produced by the activatable compound following activation by the signal generated by the signal donor. For activatable compounds that emit light, for example in the form of fluorescence, measuring the output produced by the activatable compound refers to the detection and calculation of the amount of light directly or indirectly emitted from an excited activatable compound. The output of the activatable compound can usually be measured by detecting the light (such as fluorescence) that is emitted from the activatable compound, simultaneous with or immediately following the initiation of signal production by the signal donor, regardless of whether the emission is from an excited singlet state or state of higher multiplicity.

In some embodiments, the output produced by the activatable compound is detected in serial dilutions (such as 1, 2, 3, 4, or more serial dilutions) of the sample and one or more parameters of the output are measured or calculated. The dilution series (such as number of dilutions and/or fold-dilution) is selected in order to provide a linear response, for example, where the detected signal is proportional to the concentration of the analyte. Thus, a larger-fold dilution may be required in samples containing a higher amount of analyte to provide a linear response, while samples containing a lower amount of analyte may need a smaller-fold dilution, or may even need to be concentrated to provide a linear response. In particular examples, the dilution series is determined empirically. Exemplary dilution series are described in Example 2.

In one example, the maximum slope of the signal from a dilution series is determined. The concentration of the analyte in the sample is proportional to the maximum slope, which shifts to the left (for example, compared with a control) with increasing amounts of the analyte or shifts to the right (for example, compared with a control) with decreasing amounts of the analyte. In some examples, change in the maximum slope (e.g., increase or decrease) is about 10% or more (such as at least 10%, at least 15%, or at least 20%) compared to the control. In additional examples, the amount of the analyte in the sample is further determined utilizing a standard curve or reference, for example, generated from a sample containing a known amount of the analyte.

In other examples, the maximum or peak signal from a dilution series is determined. The maximum signal is proportional to the binding affinity (K_{d}) between the analyte and the analyte-associating moiety. An increase in maximum signal (for example, compared to a control) reflects higher affinity and a decrease in maximum signal (for example, compared to a control) reflects lower affinity. In some examples, the change in the maximum signal (e.g., increase or decrease) is about 10% or more compared to the control.

In still further embodiments, if the output includes signal from two or more different activatable compounds, a ratio of the signals is determined. In one example, the ratio of signals from two different activatable compounds is used to determine if the analyte is intact or if the analyte has multiple subunits, that the subunits are assembled. A change in the ratio (for example, compared to a control) indicates that the analyte is not intact or is cleaved. In some examples, the ratio may be increased or decreased compared to a control.

In certain embodiments, the control is a sample or reference of an analyte with a known characteristic, such as known concentration, binding affinity, or structure (such as intact or fully-assembled analyte). In some examples the control or reference analyte is “wild type.” However, the control or reference analyte can be any analyte to which it is desirable to compare the sample being tested. In some examples, the control is a previous version of the analyte. For example, a variant of an analyte may be identified using the methods described herein and then go through another round of screening and selection. Thus, the variant selected in the first round may be used as the control in the second round. In other examples, the control may be a previous batch of the same material, for example for purposes of evaluating stability or consistency of the analyte and its production.

### V. Measuring DNA to Assess Host Cell Productivity

As described herein, the proximity assay can be used to determine the amount of an analyte in a sample, and in certain embodiments, the amount of active analyte can be ascertained. The amount of analyte, produced for example by a host cell culture, can be calculated as a measure of the amount of analyte produced per cell, if the relative number of host cells in that volume of culture can be determined. One way to do this is through determining the optical density (OD) of the host cell culture by measuring absorbance at a particular wavelength, for example 600 nm (OD₆₀₀). However, using OD₆₀₀ as an indication of cell number would require either a separate measurement, increasing the processing steps for the proximity assay, or the use of specialized equipment such as a plate-reader equipped to measure light scattering. However, even with specialized equipment, the detection reagents used in the proximity assay might interfere with the measurement of light scattering at approximately 600 nm as a surrogate for an absorbance determination of OD₆₀₀, and measurement of light scattering is not as sensitive as detection of the fluorescence of a DNA dye, as described below.

Another method for determining the relative number of cells in a sample, as intact or lysed cells, is to measure the nucleic acid content (e.g., DNA content) of the sample. There is a strong linear correlation between OD₆₀₀ and the DNA content of a sample (data not shown), and measuring DNA content has the advantage of being measurable in the same well by the same plate-reading apparatus that performs the proximity assay detection. An example of measuring host cell DNA content is provided as Example 2. The DNA in host cell lysate samples and in DNA standard samples can be detected with PicoGreen™ dye (ThermoFisher Scientific, Waltham, Mass.), which can be used in assays with AlphaLisa donor and acceptor beads (PerkinElmer, Waltham, Mass.) because the excitation (480 nm) and emission (520 nm) profile for PicoGreen™ dye does not interfere with the excitation and emission wavelengths used for the AlphaLisa donor and acceptor beads. Other compounds that can be used to detect DNA in the samples are propidium iodide, Hoechst 33342, 7-aminoactinomycin-D (7-AAD), and 4′6′-diamidino-2-phenylindole (DAPI).

### VI. Further Aspects of the Proximity Assay

A. Host Cell Population Genetic Diversity. The provided methods are advantageously used to select high-performing host cells (e.g., host cells expressing active analyte, properly folded analyte, and/or high expression levels of analyte) from a genetically diverse population of host cells, in which the diversity or variation within the host cell population can arise for example from differences between host cell genomes, or between expression constructs comprised by the host cells. The host cell population genetic diversity can be randomly generated by processes such as mutation, or specifically introduced by targeted methods of making changes in the host cell genome or in expression constructs, which are then introduced into the host cell strain.

The host cell population comprises a plurality of genetic variants. In many embodiments, one aspect of the disclosed methods comprises sorting a host cell population based on a predetermined property of the host cells, which predetermined property varies based on the genetic variants within the host cell population. In many embodiments, the predetermined properties of the host cells include expression level of active gene product of interest, proper folding of the gene product of interest, expression level of properly folded or intact protein, cell viability, and/or biomass. The genetic diversity of the host cell population therefore comprises a plurality of genetic variants, which genetic variants are sufficiently numerous to provide for variations in one or more of the predetermined properties within the genetically diverse population. In some embodiments, the number of genetic variants capable of substantially expressing a gene product of interest may be very small, which may require increasing the genetic diversity. Thus, in some examples, the genetic diversity of the starting host cell population may be increased until a suitable genetic diversity is achieved.

In embodiments of the disclosed methods, the genetic diversity of the host cell population is defined as the number of different genetic variants present in the host cell population, the number of different genetic variants relative to a negative control, and/or the number of different genetic variants relative to a reference cell strain. The number of genetic variants may be the actual number of variants or a calculated (“target”) number of genetic variants in the host cell population. These variants may be the result of one or more genetic (e.g., nucleic acid sequence) differences in the host cell genome between cells, one or more genetic (e.g., nucleic acid sequence) differences in expression construct(s) between host cells, or a combination thereof. In some examples, the genetic differences include alteration, deletion, or insertion of one or more nucleotides of a sequence or insertion or deletion of one or more elements (such as one or more tags, domains, expression control sequences, and/or associated protein sequences).

In some embodiments, the genetic diversity of the host cell population screened in a disclosed proximity assay is at least 100, at least 500, at least 1000, at least 1500, at least 2000, at least 3000, at least 4000, at least 5000, or at least 10,000. In other examples, the genetic diversity is about 100-10,000, such as about 100-500, about 250-1000, about 750-2000, about 1500-3000, about 2500-4000, about 3500-5000, about 4500-7500, about 6000-8000, or about 7000-10,000.

Any type of genetic diversity can be probed using the methods provided herein. In some embodiments, the genetic diversity includes one or more of differences (including alteration or presence or absence) between a gene product of interest (including but not limited to coding sequence variants and codon-optimization), promoters (including constitutive and/or inducible promoters), chaperones, ribosome binding sequences, tags, nuclear localization signals, signal peptides, knockout or knockin of one or more genes, presence of one or more (such as 1, 2, 3, or more) plasmids, or any combination thereof. In some examples, the genetic diversity is generated by standard directed genetic modification techniques. In other examples, the genetic diversity is generated by random mutagenesis, error-prone PCR mutagenesis, or transposon mutagenesis (e.g., Tn5). A combination of techniques can also be used to generate additional levels of genetic diversity.

There are many methods known in the art for making alterations to host cell genomes or expression constructs in order to change nucleotide sequences and/or to eliminate, reduce, or change gene function. Methods of making targeted disruptions of genes in host cells such as E. coli and other prokaryotes have been described (Muyrers et al., “Rapid modification of bacterial artificial chromosomes by ET-recombination”, Nucleic Acids Res 1999 Mar. 15; 27(6): 1555-1557; Datsenko and Wanner, “One-step inactivation of chromosomal genes in Escherichia coli K-12 using PCR products”, Proc Natl Acad Sci USA 2000 Jun. 6; 97(12): 6640-6645), and kits for using similar Red/ET recombination methods are commercially available (for example, the Quick & Easy E. coli Gene Deletion Kit from Gene Bridges GmbH, Heidelberg, Germany). Red/ET recombination methods can also be used to replace a promoter sequence with that of a different promoter, such as a constitutive promoter, or an artificial promoter that is predicted to promote a certain level of transcription (De Mey et al., “Promoter knock-in: a novel rational method for the fine tuning of genes,” BMC Biotechnol 2010 Mar. 24; 10: 26). The function of host cell genomes or expression constructs can also be eliminated or reduced by RNA silencing methods (Man et al., “Artificial trans-encoded small non-coding RNAs specifically silence the selected gene expression in bacteria,” Nucleic Acids Res 2011 April; 39(8): e50, Epub 2011 Feb. 3). The Gibson assembly method (Gibson, “Enzymatic assembly of overlapping DNA fragments,” Methods Enzymol 2011; 498: 349-361; doi: 10.1016/B978-0-12-385120-8.00015-2) can also be used to make targeted changes in host cell genomes or expression constructs, such as insertions, deletions, and point mutations. Another method for making directed alterations in host cell genomes or expression constructs utilizes CRISPR (clustered regularly interspaced short palindromic repeats) nucleotide sequences and Cas9 (CRISPR-associated protein 9), which recognizes and cleaves nucleotide sequences that are complementary to CRISPR sequences. Further, changes to host cell genomes can be introduced through traditional genetic methods.

In some embodiments, the genetically diverse population of host cells has been previously screened (for example, by another method) to reduce the level of genetic diversity from an initial population of host cells. In one embodiment, the population of host cells utilized in the methods described herein is obtained from cells sorted by the activity-specific cell enrichment assay described in International Application No. PCT/US2021/013734 (incorporated herein by reference in its entirety) or includes specific genetic features from cells sorted by the activity-specific cell enrichment assay.

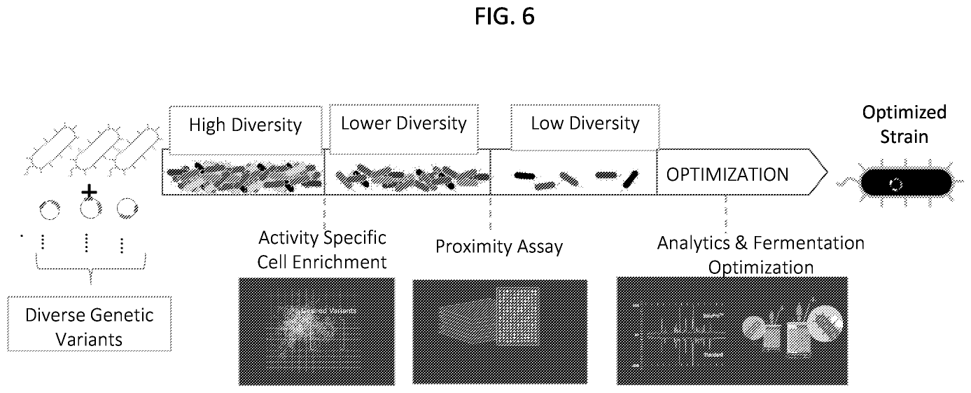

An embodiment of the activity-specific cell enrichment assay is illustrated in FIG. 6. Briefly, in some examples, prior to the methods disclosed herein, a population of host cells having a genetic diversity comprising a plurality of genetic variants, wherein at least some of the host cells comprise a polynucleotide sequence encoding a gene product of interest (such as the analyte of the present disclosure), is provided. In some examples, the method includes: culturing the population of host cells, whereby the gene product of interest is expressed by a subpopulation of the host cells of the population, the subpopulation thereby comprising expressing host cells, wherein a predetermined property of the expressing host cells varies based on the genetic variant; labeling at least some of the expressing host cells of the subpopulation, wherein the labeling comprises associating the gene product of interest with a detectable moiety, wherein an amount of the labeling is proportional to the predetermined property of the gene product of interest in the expressing host cell, thereby producing labeled expressing host cells; and selecting a subset of labeled expressing host cells, wherein the selecting comprises detecting the detectable moiety and the predetermined by a cell-sorting apparatus. In embodiments of the methods, the selecting is fluorescence-activated cell sorting. In some examples, the detectable moiety is a fluorescent moiety and the selecting comprises selecting the 0.01%-5% of cells with highest fluorescence emissions. In a particular non-limiting example, the selecting comprises selecting the 0.5% of cells with highest fluorescence emissions. In particular examples, the predetermined property of the expressing host cells comprises level of expression of active gene product of interest, level of expression of the gene product of interest, proper protein folding of the gene product of interest, level of expression of properly folded protein of the gene product of interest, cell viability, and/or amount of biomass. In additional examples, expressing host cells are determined by measuring relative expression level of the gene product of interest for each genetic variant. The selected subset of host cells, which still includes a genetic diversity, is then utilized in the methods provided herein.

B. Host Cells

Host cells of use in the disclosed methods are capable of growth at high cell density in fermentation culture, and can produce gene products in oxidizing host cell cytoplasm through highly controlled inducible gene expression. Host cells with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., “Strain engineering for improved expression of recombinant proteins in bacteria,” Microb Cell Fact 2011 May 14; 10: 32). It is also possible to express cytoplasmic forms of these Dsb proteins, such as a cytoplasmic version of DsbC (cDsbC), for example having an N-terminal truncation of twenty amino acids, which lacks a signal peptide and therefore is not transported into the periplasm. Cytoplasmic Dsb proteins such as cDsbC are useful for making the cytoplasm of the host cell more oxidizing and thus more conducive to the formation of disulfide bonds in heterologous proteins produced in the cytoplasm. The host cell cytoplasm can also be made more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (ahpC* or ahpC^{Δ}) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dup162-169 (Faulkner et al., “Functional plasticity of a peroxidase allows evolution of diverse disulfide-reducing pathways.” Proc Natl Acad Sci USA 2008 May 6; 105(18): 6735-6740, Epub 2008 May 2). (2) Optionally, host cells can also be genetically modified to express chaperones and/or cofactors that assist in the production of the desired gene product(s), and/or to glycosylate polypeptide gene products. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araF, araG, araH, rhaT, xylF, xylG, and xylH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmlA, rmlB, rmlC, and rmlD.

In certain embodiments, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli B strain 521 cells, or are E. coli B strain 522 cells. E. coli 521 and 522 cells have the following genotypes:
E. coli 521: ΔaraBAD fhuA2 [lon] ompT ahpC^{Δ} gal λatt::pNEB3-r1-cDsbC (Spec, lacI) ΔtrxB sulA11 R(mcr-73::miniTn10--Tet^{S})2 [dcm] R(zgb-210::Tn10--Tet^{S}) ΔaraEp::J23104 ΔscpA-argK-scpBC endA1 rpsL-Arg43 Δgor Δ(mcrC-mrr)114::IS10
E. coli 522: ΔaraBAD fhuA2 prpD [lon] ompT ahpC^{Δ} gal λatt::pNEB3-r1-cDsbC (Spec, lacI) ΔtrxB sulA11 R(mcr-73::miniTn10--Tet^{S})2 [dcm] R(zgb-210::Tn10--Tet^{S}) ΔaraEp::J23104 ΔscpA-argK-scpBC endA1 rpsL-Arg43 Δgor Δ(mcrC-mrr)114::IS10

In growth experiments with E. coli host cells having oxidizing cytoplasm, we have determined that E. coli B strains with oxidizing cytoplasm are able to grow to much higher cell densities than a corresponding E. coli K strain. Other suitable strains include E. coli B strains SHuffle® Express (NEB Catalog No. C3028H) and SHuffle® T7 Express (NEB Catalog No. C3029H), and the E. coli K strain SHuffle® T7 (NEB Catalog No. C3026H).

In some embodiments, the host cells are prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

Many additional types of host cells can be used in the methods provided herein, including eukaryotic cells such as yeast (Candida shehatae, Kluyveromyces lactis, Kluyveromyces fragilis, other Kluyveromyces species, Pichia pastoris, Saccharomyces cerevisiae, Saccharomyces pastorianus also known as Saccharomyces carlsbergensis, Schizosaccharomyces pombe, Dekkera/Brettanomyces species, and Yarrowia lipolytica); other fungi (Aspergillus nidulans, Aspergillus niger, Neurospora crassa, Penicillium, Tolypocladium, Trichoderma reesia); insect cell lines (Drosophila melanogaster Schneider 2 cells and Spodoptera frugiperda Sf9 cells); and mammalian cell lines including immortalized cell lines (Chinese hamster ovary (CHO) cells, HeLa cells, baby hamster kidney (BHK) cells, monkey kidney cells (COS), human embryonic kidney (HEK, 293, or HEK-293) cells, and human hepatocellular carcinoma cells (Hep G2)). The above host cells are available from the American Type Culture Collection.

C. Solid Supports. Any component(s) of the proximity assay—the analyte, the analyte-associating moiety or moieties, the signal donor, the activatable compound(s), connectors, etc. —can be attached to a solid support such as a bead, resin, or matrix, as long as that attachment does not eliminate the function of the assay component including its ability to interact with other assay components.

A solid support or a solid surface is a surface comprised of porous or non-porous water insoluble material. The surface can have any one of a number of shapes, such as strip, rod, particle, bead, and the like. The surface can be hydrophilic or capable of being rendered hydrophilic and includes inorganic powders such as silica, magnesium sulfate, and alumina; natural polymeric materials, particularly cellulosic materials and materials derived from cellulose, such as fiber-containing papers (filter paper, chromatographic paper, etc.); synthetic or modified naturally occurring polymers, such as nitrocellulose, cellulose acetate, poly(vinyl chloride), polyacrylamide, cross-linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials such as metals or glass (for example, bioglasses such as calcium sodium phosphosilicate, and ceramics). Natural or synthetic assemblies such as liposomes, lipid vesicles, and cells can also be employed. Preferably, the solid support is a particle that can be suspended in liquid, more preferably a bead comprised of a synthetic latex.

Suitable beads can be formed of particulate water-insoluble polymeric material, capable of suspension in liquid, usually having particle dimensions of 20 nm to 40 mm, and more preferably are 100 to 1000 nm in diameter. In certain embodiments the beads are a synthetic latex, such as a substituted polyethylene, examples of which include polystyrene-butadiene, polyacrylamide polystyrene, polystyrene with amino groups, polyacrylic acid, polymethacrylic acid, acrylonitrile-butadiene, styrene copolymers, polyvinyl acetate-acrylate, polyvinyl pyridine, and vinyl-chloride acrylate copolymers. Beads for use in the proximity assay of the invention can also be made of non-crosslinked polymers of styrene and carboxylated styrene, or styrene functionalized with other active groups such as amino, hydroxyl, halo, and the like; copolymers of substituted styrenes with dienes such as butadiene can also be used. In certain embodiments, beads are used as a solid support for the attachment of many molecules of the signal donor, and separate beads are used similarly for the attachment of many molecules of the activatable compound. The inclusion of a relatively large amount of signal donor and activatable compound on their respective beads can generate a larger signal from a single analyte molecule that brings a signal-donor bead and an activatable-compound bead into proximity

The binding of analytes, analyte-associating moiety or moieties, signal donors, activatable compounds, and/or other assay components to the support or surface may be accomplished by well-known techniques, commonly available in the literature (Chibata, “Immobilized enzymes: research and development,” Wiley New York 1978; and Cuatrecasas, “Protein purification by affinity chromatography: derivatizations of agarose and polyacrylamide beads,” J Biol Chem 1970 June; 245(12): 3059-3065). The surface will usually be polyfunctional or be capable of being polyfunctionalized or be capable of binding ligands, signal donors, and activatable compounds through specific or non-specific covalent or non-covalent interactions. A wide variety of functional and/or linking groups are available or can be incorporated. Functional groups include carboxylic acids, aldehydes, amino groups, cyano groups, ethylene groups, hydroxyl groups, and mercapto groups. The length of a linking group may vary widely, depending upon the nature of the compound being linked, and the effect on specific binding properties of the distance between the compound being linked and the surface.

D. High-Throughput Capability. The aliquoting and handling of samples in the disclosed methods can be automated in a number of ways known to one of ordinary skill in the art. For example, the use of multiwell plates, robotic dispensing and autosampling devices, and plate-readers creates a high-throughput process capable of handling 10,000 or more samples per assay cycle. Mechanisms suitable for use in the provided assays include those that accurately dispense volumes as small as 0.001 mL or lower (for example, volumes as small as 25 nL), including liquids containing suspensions of solid support beads, and those that accommodate multiwell plates, such as plates with 6, 12, 24, 48, 96, 384, 1536, 3456, or 9600 wells. The assay can be carried out in microfluidic devices; all of the assay components, including host cells and solid support beads, can have diameters within the microfluidic device channel size range of 100 nm to 0.5 mm

In some embodiments, the disclosed assays can be used to rapidly screen large numbers of samples, such as a population of host cells with a high level of genetic diversity. In some examples, the assay can be used to rapidly screen about 96 samples, about 384 samples, about 1536 samples, about 3456 samples, about 9600 samples, or more. For example, the disclosed assays can be used to screen about 4800 samples per day, about 9600 samples per day, about 14,400 samples per day, about 19,200 samples per day, or more. In other examples, the disclosed assays are able to screen samples at a rate of about 1-3 seconds per sample. The samples may be an individual host cell sample, replicates of a host cell sample, serial dilutions of a host cell sample, or a combination thereof.

E. Use of Proximity Assay with Other Assays. The proximity assay described herein can be used in conjunction with other assays and/or certain purification procedures for the purpose of characterizing the analyte. For example, the proximity assay can be a means of identifying samples for further testing by solid-phase extraction mass spectrometry (SPE-MS), or other methods described in Example 3. In other instances it could be desirable to purify the analyte to some degree before performing the proximity assay, although as shown in Examples 1 and 2, such purification is not usually necessary. Examples of other assays and procedures that can be performed on the analyte include bio-layer interferometry, DNA sequencing, enzyme-linked immunosorbent assay (ELISA), gel electrophoresis, immunofluorescence staining, surface plasmon resonance, and liquid chromatography (LC) including affinity chromatography and high-performance liquid chromatography (HPLC).

### EXAMPLES

The following examples are provided to illustrate certain particular features and/or embodiments. These examples should not be construed to limit the disclosure to the particular features or embodiments described.

### Example 1

### Use of Proximity Assay to Detect Active Trastuzumab

### A. Production of Trastuzumab, Proximity Assay Protocol, and Results

Trastuzumab, also referred to by its trade name Herceptin®, is a full-length humanized IgG1 monoclonal antibody that recognizes the HER2 antigen, also called ERBB2. The amino acid sequences of the heavy chain and the light chain of trastuzumab are provided as SEQ ID NOs: 1 and 2, respectively. These amino acid sequences lack signal peptides and are intended to be expressed in the host cell cytoplasm.

A dual-promoter expression vector was prepared comprising coding sequences for the heavy chain and the light chain of trastuzumab, with the heavy chain of trastuzumab expressed from an L-arabinose-inducible promoter, and the light chain of trastuzumab expressed from a propionate-inducible promoter. This expression vector was transformed into an E. coli host cell line. The host cells were grown in a 150-mL fermentation culture, and expression of the trastuzumab heavy and light chains were induced with varying amounts of L-arabinose and propionate, respectively, as shown in Table 1. Following the induction period, the host cells were harvested, and the pellets saved for analysis to determine the amount of trastuzumab produced by the host cells. The host cell pellets that were saved for the following experiments correspond to 0.005 mL of culture volume, and host cells present at an optical density at 600 nm (OD₆₀₀) that can vary between about 70 and about 205. As shown in Table 1, each of the samples had an OD₆₀₀ between 80 and 85 when harvested. The host cell pellets were stored at ˜80 degrees C.

To determine the amount of fully assembled and active trastuzumab produced by the host cells, the host cells were lysed, and the trastuzumab present in the lysate was contacted with detection reagent. It is recommended that electrophilic protease inhibitors not be added to the host cell lysate, because the presence of electrophilic protease inhibitors in the lysate has been found to suppress the binding of a detection reagent to the antigen-binding sites of trastuzumab, presumably due to effects of the protease inhibitors on the nucleophilic serine residues of the trastuzumab complementarity-determining region (CDR). The detection reagent comprised biotinylated HER2 protein, signal-donor (or “donor”) beads coated with streptavidin, and activatable-compound (or “acceptor”) beads coated with streptavidin. When the HER2 protein in this detection system binds to a trastuzumab antigen-binding site, it can be connected to either a donor bead, or to an acceptor bead, through the interaction of the biotin molecule covalently attached to HER2 and a streptavidin molecule attached to the bead. In this assay, a detectable output should only be produced by the acceptor bead when it is bound through HER2 to one antigen-binding site, and there is a donor bead bound through HER2 to the other antigen-binding site of a fully assembled and active trastuzumab antibody, creating the required proximity between the donor and acceptor beads (see FIG. 2).

The specific proximity assay protocol that was used to measure trastuzumab is as follows. The frozen host cell pellets were thawed on ice for five minutes, then 0.1 mL of GLB complete lysis buffer was added to each pellet. GLB complete lysis buffer is 50 mM Tris pH 7.4, 200 mM NaCl, 216 U benzonase, 1% octylglucoside, and 240 kU rLysozyme per 100 mL. Although the presence of octylglucoside can inhibit the detectable output produced by the AlphaLisa detection reagents, once the lysate has been diluted at least 20-fold, the inhibitory effect of this detergent is minimal (data not shown). The pellet and lysis buffer were mixed by vortexing and/or pipetting for 30 seconds, and the mixture was incubated on ice for 10 to 30 minutes. Serial dilutions were made of the resulting lysates in 96-well plates. In column 1 of the plate, 0.2 mL of Immunoassay Buffer (25 mM HEPES pH 7.4, 0.1% Casein, 1 mg/mL Dextran-500, 0.5% Triton X-100, and 0.05% Proclin-300) was dispensed, and in columns 2-12, 0.026 mL of Immunoassay Buffer was dispensed. Dilution 1: adding 0.005 mL of the resuspended lysate (already diluted 1:20 relative to the host cell culture) to the 0.2 mL of Immunoassay Buffer in the first column, and mixing well by pipetting, resulting in a 1:820 dilution. Dilution 2: transferring 0.040 mL of the first dilution into the 0.026 mL of Immunoassay Buffer in the second column, and mixing well by pipetting, resulting in a 1:1353 dilution. Dilutions 3-12: repeating the steps for Dilution 2 in columns 3-12 across the plate; the final dilution is an approximately 1:202,366 dilution. Serial dilutions of two standard samples of trastuzumab were also prepared, made in the same way as for the experimental samples, but starting with a standard solution of trastuzumab. The most concentrated trastuzumab standard dilution samples were 6 nM, and the least concentrated trastuzumab standard dilution samples were approximately 24 pM. The proximity assay when performed as follows is intended to measure analytes at concentrations approximately between 50 pM and 1 nM.

The detection reagent was prepared by aliquoting the appropriate amounts of stock preparations of biotinylated HER2 (SPEED BioSystems, Gaithersburg, Md.), streptavidin-coated AlphaLisa donor and acceptor beads (PerkinElmer, Waltham, Mass.), and 10× Immunoassay Buffer (250 mM HEPES pH 7.4, 1% Casein, 10 mg/mL Dextran-500, 5% Triton X-100, and 0.5% Proclin-300, available from PerkinElmer, Waltham, Mass.) to create a detection reagent solution that is 1.5× (1.66 nM) biotinylated HER2, and 1.5× (30 mg/L) streptavidin donor beads and acceptor beads in 1× Immunoassay Buffer. The detection reagent was dispensed into the wells of a 384-well plate, 0.012 mL per well, and a 0.006 mL sample from each well in the lysate dilution series was added to a well in the 384-well plate. The 384-well plate was sealed with a clear plastic plate seal (designed for real-time PCR) and was covered with a light-proof lid, then sandwiched between a blank 384-well plate on the top and bottom (to prevent condensation on the seal), and incubated at 4 degrees C. overnight. After the plate had warmed back up to room temperature by sitting at room temperature for about an hour, the results were read using an EnSpire Multimode plate reader (PerkinElmer, Waltham, Mass.), with excitation by a laser pulse at 680 nm, and the emissions detected at 620 nm.

The emissions readings from the serial dilutions of the host cell lysate samples and of the averaged reading for the serial dilutions of the trastuzumab standards were plotted as curves, and the slopes of the lines between each segment of the curves were calculated. The maximum slope for each host cell lysate sample was divided by the maximum slope for the serial dilutions of the trastuzumab standard, and multiplied by the known concentration of the trastuzumab standard, to determine the trastuzumab concentration in each of the host cell lysate samples. The concentrations of active trastuzumab for each sample, as determined by the proximity assay, are shown in Table 1.

TABLE 1
Quantification of Trastuzumab by Proximity Assay

| | | | Optical | Proximity |
|---|---|---|---|---|
| | | | Density | Assay: Active |
| | [L-arabinose] | [propionate] | (OD600) | Trastuzumab |
| Bioreactor | (mM) | (mM) | at Harvest | (g/L) |
| 1 | 21 | 336 | 84.8 | 0.319 |
| 2 | 21 | 0 | 80.0 | 0.167 |
| 3 | 21 | 21 | 81.6 | 0.213 |
| 4 | 21 | 168 | 84.4 | 0.300 |

### B. Quantification of Trastuzumab Tetramer by ProA-Affinity Purification and Size-Exclusion Chromatography (SEC)

The trastuzumab protein obtained from the pelleted host cells was also quantified using a ProA purification step followed by size-exclusion chromatography (SEC). In this method, referred to as ProA SEC, three duplicate host cell pellets were analyzed that were prepared from the same fermentation cultures as the host cell pellets analyzed by the proximity assay. The pellets, corresponding to 0.1 mL of host cell culture volume, were thawed at room temperature for 10 minutes, and 1 mL of GLB complete lysis buffer with 1% octylglucoside was added to each host cell pellet. The samples were vortexed vigorously and incubated on ice for 20 to 30 minutes, followed by centrifugation at 20,000×g for 60 minutes at 4 degrees C.

The ProA purification step used 0.04 mL of a 50% slurry of MabSelect™ (GE Healthcare Life Sciences, Marlborough, Mass.) ProA affinity medium in PBS buffer, aliquoted into an Eppendorf tube for each sample, to which was added 0.7 mL of the host cell lysate. The samples were incubated at 4 degrees C. on a rotator running at 20 rpm for 60 to 90 minutes, then transferred into the wells of a 96-well 1-mL 0.45-micrometer pore AcroPrep™ vacuum-filtration plate (Pall, Port Washington, New York). The liquid in the sample wells was removed by vacuum filtration, the samples were washed three times by addition of 0.2 mL 1×PBS followed by mixing with a pipette tip and vacuum filtration, then the samples were eluted twice, each time by adding 0.2 mL of 100 mM glycine pH 3.0 to each well, followed by centrifugation of the plate at 500×g for 2 minutes at 4 degrees C., with the well contents centrifuged into a collection plate containing 0.02 mL 0.5 M MES (2-(N-morpholino)ethanesulfonic acid). For the HP-SEC analysis of the protein purified by ProA, 0.025 mL of the eluate was loaded onto a Zenix-C SEC-300 column (Sepax Technologies, Newark, Del.), equilibrated with 150 mM sodium phosphate pH 7.0, eluted isocratically at 1 mL per minute for 20 minutes, with detection by absorbance at 280 nm. The peak retention time for trastuzumab tetramer was approximately 7.8 minutes.

To determine the amount of trastuzumab tetramer produced by the host cells, the total amount of protein affinity-purified with ProA was determined by measuring the scatter-corrected 280-nm absorbance (subtracting the blank-corrected 320-nm absorbance from the blank-corrected 280-nm absorbance) for each sample. The scatter-corrected 280-nm absorbance was divided by the extinction coefficient, which is 1.48 L/g·cm, resulting in the ProA eluate concentration expressed in g/L, which was multiplied by the ProA eluate volume (220 mL) to obtain the total amount (g) of protein purified by ProA, and that value was divided by the amount of culture volume (0.07 mL) loaded onto the ProA resin, and the results among the three duplicate samples that were tested were averaged, to result in the (average) total ProA protein yield per host cell culture volume (g/L, see Table 2). The percentage of that total ProA protein yield that is trastuzumab tetramer was determined by the SEC analysis; multiplication of the total ProA protein yield by the percentage of trastuzumab tetramer produces the amount of ProA SEC trastuzumab tetramer in g/L of host cell culture volume (see Table 2). In addition to these results of the quantitation of trastuzumab protein by ProA SEC, Table 2 also includes the quantities determined by the proximity assay for comparison. The amount of active trastuzumab as measured by the proximity assay is somewhat less in these samples than the amount as measured by ProA SEC. This result could be due to inherent differences between the two assays.

TABLE 2
Quantification of Trastuzumab by ProA SEC and Proximity Assay

| | | ProA SEC: | |
|---|---|---|---|
| | | Trastuzumab | Proximity |
| | | Tetramer | Assay: Active |
| | ProA Total | (% of ProA total | Trastuzumab |
| Bioreactor | Protein (g/L) | protein, g/L) | (g/L) |
| 1 | 0.681 | 61.7%, 0.420 | 0.319 |
| 2 | 0.548 | 54.6%, 0.299 | 0.167 |
| 3 | 0.770 | 55.6%, 0.428 | 0.213 |
| 4 | 0.830 | 58.1%, 0.482 | 0.300 |

### C. Further Comparison of Proximity Assay to ProA (SEC) Quantification Methods

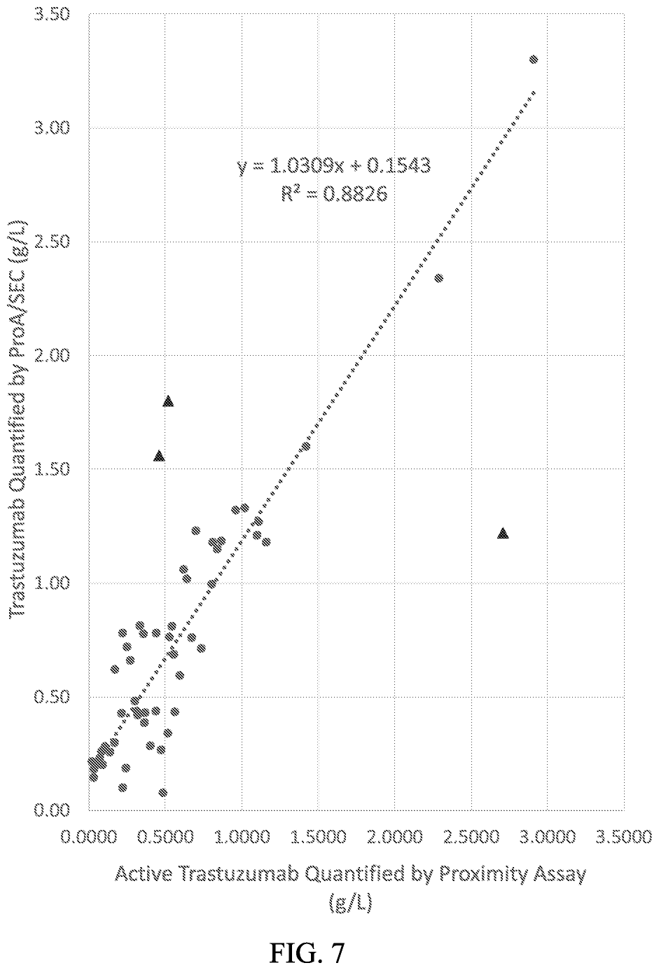

The proximity assay and ProA SEC were used to determine the amount of trastuzumab in many different lysate samples, including those described in Example 1A. The procedures for creating the host cell lysates comprising trastuzumab were similar to that described in Example 1A, with the use of varying expression construct sequences and culture and induction conditions. A comparison of the quantities of trastuzumab determined by these two methods for all of these sample is shown in FIG. 7. As can be seen, there is a strong correlation between the quantities determined by the two different assays: the value calculated for the correlation coefficient, when the indicated outlier data points are removed, is 0.939457. In these experiments the proximity assay, which measures active trastuzumab, almost always produced a somewhat lower result than the ProA SEC assay, which measures ProA-purified trastuzumab tetramer. Other than one of the outlier points, the only data points where the proximity assay produced a higher result than the ProA SEC assay were at lower trastuzumab concentrations (see the lower left portion of the graph in FIG. 7), where a higher relative level of error in the assays is expected due to a lower signal-to-noise ratio.

### Example 2

### Proximity Assay with DNA Staining to Normalize Results for Cell Number

The proximity assay, with the addition of a dye for DNA staining, can be carried out as follows.

1. If necessary, thaw a frozen host cell pellet on ice for five minutes.

2. Resuspend the host cell pellet in Dilution Buffer I: 1×PBS, 1 mM EDTA, 0.1× Immunoassay Buffer (2.5 mM HEPES pH 7.4, 0.01% Casein, 0.1 mg/mL Dextran-500, 0.05% Triton X-100, and 0.005% Proclin-300). For example, a host cell pellet corresponding to 0.05 mL host cell culture is resuspended in 0.2 mL Dilution Buffer I, for an initial four-fold dilution relative to the host cell culture.

3. A four-step dilution series of the host cells is made prior to lysis, with the dilution factor depending on the anticipated amount of the analyte being produced in the host cells: a higher dilution factor is used for analytes produced at a high concentration per cell (for example, a further 25-fold dilution of the 1:4 host cell resuspension, and then three 12-fold dilution steps to generate a dilution range from 1:100 to 1:172,800), and a smaller dilution factor is used for analytes produced at a moderate concentration per cell (for example, four 8-fold dilution steps to generate a range from 1:32 to 1:16,384). For analytes that are produced at a low level per cell, and/or for analytes with a lower level of detectable output produced by the proximity assay, the harvested host cells can be concentrated, for example to five times the concentration that was present in the host cell culture, then diluted in four 5-fold dilution steps to generate a range from 1:1 to 1:125. The dilution series is made in a 384-well deep-well plate, with the host cell solution transferred at each dilution step into wells containing Dilution Buffer II: 1 mM EDTA and 1× Immunoassay Buffer (25 mM HEPES pH 7.4, 0.1% Casein, 1 mg/mL Dextran-500, 0.5% Triton X-100, and 0.05% Proclin-300). A four-step dilution series is also created for a standard solution of the analyte at a known concentration, with the highest concentration of the standard usually not exceeding 10 nM, so that the serial dilution range of the standard overlaps with the optimal assay range which is approximately between 50 pM and 1 nM. A DNA standard dilution series can also be created, for example, starting with a DNA solution that has been diluted 1:25 from 0.1 mg/mL to 4000 ng/mL in 1 mM EDTA and 0.1× Immunoassay Buffer, then four 1:5 dilution steps in 1 mM EDTA and 0.1× Immunoassay Buffer, resulting in the lowest concentration of 32 ng/mL. It is also possible to compare the signal generated by DNA in test samples to the signal generated by DNA in samples derived from a control, such as a positive control host cell strain, to identify host cell cultures that produce relatively more analyte per cell than the control host cell strain.

4. For some analytes and combinations of detection reagents, lysozyme (for analytes produced in host cell cytoplasm), the analyte-associating moiety or moieties, the signal donor, the activatable compound(s), and a dilution of host cells comprising the analyte can all be combined in an assay well in one step, so that cell lysis and the detection of the analyte occur together. However, certain analytes and/or detection reagents generate more output for detection when combined in two steps as described below, possibly due to an inhibitory steric effect of donor and acceptor beads on the interaction between the analyte and the analyte-associating moiety or moieties.

A 384-well shallow-well assay plate is prepared by dispensing 0.006 mL of Assay/Lysis Buffer I (1 mM EDTA, lx Immunoassay Buffer, 240 kU rLysozyme per 100 mL if needed, analyte-associating moiety or moieties at effective concentrations, and PicoGreen™ dye (ThermoFisher Scientific, Waltham, Mass.) at a dilution of 1:200) into each well. This combination of 1 mM EDTA and lysozyme is sufficient for lysis of host cells; detergents such as octylglucoside are preferably not used because many detergents will inhibit the production of detectable output by the AlphaLisa reagents. A 0.006 mL aliquot of each dilution of the host cells is transferred into a well of the assay plate, and incubated at room temperature for an hour to allow for lysis and the association of the analyte with the analyte-associating moiety or moieties. Following this incubation, 0.006 mL of Assay Buffer II (1 mM EDTA, lx Immunoassay Buffer, and the signal donor and the activatable compound—for example, streptavidin donor and acceptor beads at 60 mg/L) is dispensed into each well, and the plate is sealed, incubated for 1 to 24 hours at 4 degrees C., warmed to room temperature, read, and the data analyzed, generally as described in Example 1A. In addition to the reading of emissions related to the samples, the plate is read with an excitation of 480 nm and at an emission of 520 nm to measure the DNA content. If the plate is initially read following a relatively short incubation (one to two hours, for example), it can be read again after a longer incubation period, as the detectable output from the acceptor bead continues to increase until the maximum detectable output is achieved at equilibrium, typically after approximately 16 to 22 hours of incubation. When calculating the maximum slopes for the dilution curves generated for the test samples and for the standards, as described in Example 1A, it can be the case that the standard has been diluted to a different degree than the test samples. In that situation, to generate the dilution curves, the dilution-adjusted actual volume is plotted on one axis (such as the x-axis) versus the detected output on the other axis (such as the y-axis), and the maximum slopes are calculated from those curves. The slopes of the lines for the test samples and for the standards will have the same units (signal/dilution-adjusted actual volume), and the calculation method described in Example 1A will give the desired result.

### Example 3

### Characterizing Disulfide Bonds

The number and location of disulfide bonds in polypeptide analytes can be determined by digestion of the polypeptide analyte with a protease, such as trypsin, under non-reducing conditions, and subjecting the resulting peptide fragments to mass spectrometry (MS) combining sequential electron transfer dissociation (ETD) and collision-induced dissociation (CID) MS steps (MS2, MS3) (Nili et al., “Defining the disulfide bonds of insulin-like growth factor-binding protein-5 by tandem mass spectrometry with electron transfer dissociation and collision-induced dissociation,” J Biol Chem 2012 Jan. 6; 287(2): 1510-1519; Epub 2011 Nov. 22).

Digestion of coexpressed protein. To prevent disulfide bond rearrangements, free cysteine residues are blocked by alkylation: the polypeptide analyte is incubated protected from light with the alkylating agent iodoacetamide (5 mM) with shaking for 30 minutes at 20 degrees C. in buffer with 4 M urea, and then is separated by non-reducing SDS-PAGE using precast gels. Alternatively, the polypeptide analyte is incubated in the gel after electrophoresis with iodoacetamide, or without as a control. Protein bands are stained, de-stained with double-deionized water, excised, and incubated twice in 0.5 mL of 50 mM ammonium bicarbonate, 50% (v/v) acetonitrile while shaking for 30 minutes at 20 degrees C. Protein samples are dehydrated in 100% acetonitrile for 2 minutes, dried by vacuum centrifugation, and rehydrated with 10 mg/ml of trypsin or chymotrypsin in buffer containing 50 mM ammonium bicarbonate and 5 mM calcium chloride for 15 minutes on ice. Excess buffer is removed and replaced with 0.05 mL of the same buffer without enzyme, followed by incubation for 16 hours at 37 degrees C. or at 20 degrees C., for trypsin and chymotrypsin, respectively, with shaking. Digestion is stopped by adding 3 microliters of 88% formic acid, and after brief vortexing, the supernatant is removed and stored at ˜20 degrees C. until analysis.

Localization of disulfide bonds by mass spectrometry. Peptides are injected onto a 1 mm×8 mm trap column (Michrom BioResources, Inc., Auburn, Calif.) at 20 microliters/minute in a mobile phase containing 0.1% formic acid. The trap cartridge is then placed in-line with a 0.5 mm×250 mm column containing 5 mm Zorbax SB-C18 stationary phase (Agilent Technologies, Santa Clara, Calif.), and peptides separated by a 2-30% acetonitrile gradient over 90 minutes at 10 micro-liters/minute with a 1100 series capillary HPLC (Agilent Technologies). Peptides are analyzed using an LTQ Velos linear ion trap with an ETD source (Thermo Scientific, San Jose, Calif.). Electrospray ionization is performed using a Captive Spray source (Michrom Bioresources, Inc.). Survey MS scans are followed by seven data-dependent scans consisting of CID and ETD MS2 scans on the most intense ion (e.g., the ion (m/e) having the most intense peak through the scanned m/e range) in the survey scan, followed by five MS3 CID scans on the first- to fifth-most intense ions in the ETD MS2 scan. CID scans use normalized collision energy of 35, and ETD scans use a 100 ms activation time with supplemental activation enabled. Minimum signals to initiate MS2 CID and ETD scans are 10,000, minimum signals for initiation of MS3 CID scans are 1000, and isolation widths for all MS2 and MS3 scans are 3.0 mk. The dynamic exclusion feature of the software is enabled with a repeat count of 1, exclusion list size of 100, and exclusion duration of 30 s. Inclusion lists to target specific crosslinked species for collection of ETD MS2 scans are used. Separate data files for MS2 and MS3 scans are created by Bioworks 3.3 (Thermo Scientific) using ZSA charge state analysis. Matching of MS2 and MS3 scans to peptide sequences is performed by Sequest (V27, Rev 12, Thermo Scientific). The analysis is performed without enzyme specificity, a parent ion mass tolerance of 2.5, fragment mass tolerance of 1.0, and a variable mass of +16 for oxidized methionine residues. Results are then analyzed using the program Scaffold (V3_00_08, Proteome Software, Portland, Oreg.) with minimum peptide and protein probabilities of 95 and 99% being used. Peptides from MS3 results are sorted by scan number, and cysteine containing peptides are identified from groups of MS3 scans produced from the five most intense ions observed in ETD MS2 scans. The identities of cysteine peptides participating in disulfide-linked species are further confirmed by manual examination of the parent ion masses observed in the survey scan and the ETD MS2 scan.

### Embodiments

In addition to, or as an alternative to the above, the following embodiments are described:

Embodiment 1 is directed to a method for determining the presence or amount of an analyte in a sample that is in the active form of the analyte, the method comprising:

(i) combining the sample comprising the analyte, a first complex comprising a signal donor and a first analyte-associating moiety, and a second complex comprising an activatable compound and a second analyte-associating moiety;

wherein the sample comprises active analyte that is in the active form of the analyte and inactive analyte that is not in the active form of the analyte; and

wherein the active analyte associates with both the first complex and the second complex, and the inactive analyte does not associate with both the first complex and the second complex;

(ii) initiating the transfer of a signal from the signal donor in the first complex, wherein the signal is received by the activatable compound in the second complex that is associated with an active analyte associated with both the first complex and the second complex; and

(iii) detecting the output from the activatable compound.

Embodiment 2 is directed to a method for determining the presence or amount of an analyte in a sample, the method comprising:

(i) contacting the analyte with a first complex comprising a signal donor and a first analyte-associating moiety, and a second complex comprising an activatable compound and a second analyte-associating moiety;

(ii) initiating the transfer of a signal from the signal donor to the activatable compound, wherein the signal causes the activatable compound to produce detectable output; and

(iii) detecting the output from the activatable compound.

Embodiment 3 is directed to the method of embodiments 1 or 2, further comprising:

contacting the analyte with a third complex comprising a second activatable compound and a third analyte-associating moiety;

wherein the transfer of the signal from the signal donor to the second activatable compound causes the second activatable compound to produce detectable output; and

detecting the output from the second activatable compound.

Embodiment 4 is directed to a method for determining the presence or amount of an analyte in a sample, the method comprising:

(i) providing a covalently labeled analyte that comprises the analyte connected through a covalent bond to a first detection reagent selected from the group consisting of a signal donor and an activatable compound;

(ii) contacting a sample comprising the analyte with the covalently labeled analyte and with an analyte-associating moiety connected to a second detection reagent selected from the group consisting of a signal donor and an activatable compound, wherein the first detection reagent is not the second detection reagent;

(iii) initiating the transfer of a signal from the signal donor to the activatable compound, wherein the signal causes the activatable compound to produce detectable output; and

(iv) detecting the output from the activatable compound.

Embodiment 5 is directed to the method of any one of embodiments 2 to 4, wherein the sample comprises active analyte that is in the active form of the analyte and inactive analyte that is not in the active form of the analyte, wherein the active analyte associates with both the first complex and the second complex, and the inactive analyte does not associate with both the first complex and the second complex.

Embodiment 6 is directed to the method of any one of embodiments 1 to 5, wherein the sample comprises a population of host cells having a genetic diversity, the genetic diversity comprising a plurality of genetic variants, wherein at least some of the population of host cells comprise a polynucleotide encoding the analyte.

Embodiment 7 is directed to the method of embodiment 6, wherein the genetic diversity of the host cell population is host cell genomic variation, polynucleotide sequence variation of one or more expression constructs, or a combination thereof, comprised by at least some of the host cells of the host cell population.

Embodiment 8 is directed to the method of embodiments 6 or 7, wherein the genetic diversity of the population of host cells is about 1500 or about 5000.

Embodiment 9 is directed to the method of any one of embodiments 6 to 8, further comprising selecting the population of host cells having genetic diversity and encoding the analyte prior to contacting the sample and/or the analyte with one or more of an analyte-associating moiety, the signal donor, and the activatable compound(s).

Embodiment 10 is directed to the method of embodiment 9, wherein selecting the population of host cells having genetic diversity and encoding the analyte comprises:

culturing a population of host cells, whereby the analyte is expressed by a subpopulation of the host cells of the population, the subpopulation thereby comprising expressing host cells;

labeling at least some of the expressing host cells of the subpopulation, wherein the labeling comprises associating the gene product of interest with a detectable moiety, thereby producing labeled expressing host cells; and

selecting a subset of labeled expressing host cells, wherein the selecting comprises detecting the detectable moiety by a cell-sorting apparatus.

Embodiment 11 is directed to the method of any one of embodiments 1 to 10, wherein the analyte comprises a polypeptide, a protein, a glycoprotein, a phosphoprotein, a proteolipid, a polypeptide comprising one or more domains or fragments of any of the preceding, and a nucleic acid.

Embodiment 12 is directed to the embodiment of embodiment 11, wherein the analyte is selected from the group consisting of adhesins, antibodies, antigens, cytokines, enzymes, growth factors, ligands, receptors, structural proteins, transcription factors, transporter proteins, polypeptide toxins, protein toxins, and polypeptides comprising one or more domains or fragments of any of the preceding

Embodiment 13 is directed to the method of embodiment 12, wherein the analyte is an antibody or a polypeptide comprising an antibody domain.

Embodiment 14 is directed to the method of embodiment 13, wherein the analyte comprises an antigen-binding domain Embodiment 15 is directed to the method of embodiment 14, wherein the analyte is a monoclonal antibody and/or a bispecific antibody.

Embodiment 16 is directed to the method of any one of embodiments 1 to 15, wherein the analyte is a homomultimer and/or the analyte has properly formed disulfide bonds.

Embodiment 17 is directed to the method of any one of embodiments 1 to 16, wherein the analyte is a homomultimer and the analyte-associating moiety of the first complex is the same as the analyte-associating moiety of the second complex.

Embodiment 18 is directed to the method of anyone of embodiments 1 to 17, wherein:

the analyte comprises an antigen-binding domain and the analyte-associating moiety is an antigen that binds to the antigen-binding domain of the analyte; and/or

the analyte-associating moiety comprises an antigen-binding domain and the analyte is an antigen that binds to the antigen-binding domain of the analyte-associating moiety; and/or

the analyte comprises a ligand-binding domain and the analyte-associating moiety is a ligand that binds to the ligand-binding domain of the analyte; and/or

the analyte-associating moiety comprises a ligand-binding domain and the analyte is a ligand that binds to the ligand-binding domain of the analyte-associating moiety; and/or

the analyte comprises a domain with enzymatic activity and the analyte-associating moiety is a substrate that binds to the domain with enzymatic activity of the analyte; and/or

the analyte-associating moiety comprises a domain with enzymatic activity and the analyte is a substrate that binds to the domain with enzymatic activity of the analyte-associating moiety; and/or

the analyte-associating moiety is an antibody that specifically binds to the analyte; and/or the analyte is an antibody that specifically binds to the analyte-associating moiety.

Embodiment 19 is directed to the method of any one of embodiments 1 to 18, further comprising contacting an assay component with a first antibody that specifically binds the assay component, where the assay component is selected from the group consisting of the analyte and the analyte-associating moiety.

Embodiment 20 is directed to the method of embodiment 19, further comprising contacting the first antibody with a second antibody that specifically binds the first antibody.

Embodiment 21 is directed to the method of embodiment 20, wherein the second antibody is an anti-species antibody.

Embodiment 22 is directed to the method of any one of embodiments 1 to 21, wherein the analyte-associating moiety is a multimer that can interact with the analyte at multiple sites on the analyte.

Embodiment 23 is directed to the method of any one of embodiments 1 to 22, wherein the analyte-associating moiety is attached to a detection reagent selected from the group consisting of a signal donor and an activatable complex.

Embodiment 24 is directed to the method of embodiment 23, wherein the analyte-associating moiety is attached to the detection reagent through a connector,

Embodiment 25 is directed to the method of embodiment 24, wherein the connector comprises a polypeptide linker and/or a binding pair.

Embodiment 26 is directed to the method of embodiment 25, wherein the binding pair is biotin and streptavidin, or the binding pair is polyhistidine and a metal ion selected from the group consisting of nickel ion (Ni²) and cobalt ion (Co²±).

Embodiment 27 is directed to the method of any one of embodiments 1 to 26, wherein the signal donor is activated by an enzyme and/or the signal donor is activated by irradiation.

Embodiment 28 is directed to the method of any one of embodiments 1 to 27, wherein the signal donor produces a fluorescence resonance transfer signal, and/or the signal donor produces a chemical signal.

Embodiment 29 is directed to the method of embodiment 28, wherein the chemical signal is a reactive oxygen species.

Embodiment 30 is directed to the method of embodiment 29, wherein the reactive oxygen species is singlet oxygen.

Embodiment 31 is directed to the method of any one of embodiments 1 to 30, wherein the signal donor is a sensitizer.

Embodiment 32 is directed to the method of embodiment 31, wherein the sensitizer is a haloperoxidase.

Embodiment 33 is directed to the method of embodiment 31, wherein the signal donor is a photosensitizer.

Embodiment 34 is directed to the method of embodiment 33, wherein the signal donor is a photosensitizer with an absorption maximum in the wavelength range of 250-1100 nm, and/or 300-1000 nm, and/or 450-950 nm, and/or is a photosensitizer with an extinction coefficient at its absorbance maximum in the range of 500 M-1cm-1 to 100,000 M-1cm-1, and/or in the range of 5,000 M-1cm-1 to 100,000 M-1cm-1; and/or in the range of 50,000 M-1cm-1 to 100,000 M-1cm-1

Embodiment 35 is directed to the method of embodiments 33 or 34, wherein the photosensitizer is selected from the group consisting of ketones, xanthenes, polyaromatic compounds, porphyrins, oxazines, squarate dyes, cyanines, and thiazines; and/or the photosensitizer is selected from the group consisting of benzophenone, 9-thioxanthone, eosin, rose bengal, buckminsterfullerene, 9,10-dibromoanthracene, hematoporphyrin, chlorophylis, a phthalocyanine, a naphthalocyanine, a merocyanine, and methylene blue.

Embodiment 36 is directed to the method of any one of embodiments 1 to 35, wherein the activatable compound is a photoactivable compound.

Embodiment 37 is directed to the method of embodiment 36, wherein the photoactivable compound emits light by fluorescence.

Embodiment 38 is directed to the method of embodiment 37, wherein the photoactivable compound with an emission quantum yield between 0.05 and 1.0, and/or between 0.1 and 1.0, and/or between 0.4 and 1.0, and/or between 0.7 and 1.0.

Embodiment 39 is directed to the method of any one of embodiments 36 to 38, wherein the photoactivable compound is selected from the group consisting of xanthenes, bimanes, coumarins, aromatic amines, squarate dyes, benzofurans, cyanines, rare earth chelates, porphyrins, polyaromatic compounds, and chromenes.

Embodiment 40 is directed to the method of embodiment 39, wherein the photoactivable compound is selected from the group consisting of rhodamine, fluorescein, umbelliferone, dansyl, a merocyanine, a phthalocyanine, pyrene, anthracene, and acenaphthene.

Embodiment 41 is directed to the method of any one of embodiments 36 to 39, wherein the photoactivable compound undergoes a chemical reaction with singlet oxygen.

Embodiment 42 is directed to the method of embodiment 41, wherein the photoactivable compound that undergoes a chemical reaction with singlet oxygen emits light within the wavelength range of 250 to 1200 nm, and/or 300 to 1200 nm, and/or 500 to 1200 nm, and/or 600 to 1200 nm, and/or 600 to 800 nm.

Embodiment 43 is directed to the method of embodiments 41 or 42, wherein the photoactivable compound that undergoes a chemical reaction with singlet oxygen is selected from the group consisting of enol ethers, enamines, 9-alkylidene-N-alkylacridans, arylvinylethers, dioxenes, arylimidazoles, 9-alkylidene-xanthanes, and lucigenin.

Embodiment 44 is directed to the method of any one of embodiments 1 to 43, further comprising measuring the optical density of the sample.

Embodiment 45 is directed to the method of embodiment 44, wherein, the optical density of the sample is measured at 600 nm.

Embodiment 46 is directed to the method of embodiment 44, wherein the optical density of the sample is measured by detecting light scattering.

Embodiment 47 is directed to the method of any one of embodiments 1 to 46, further comprising adding a compound that interacts with nucleic acid to the sample, irradiating the sample to excite the compound that interacts with nucleic acid, and measuring the light emitted by the excited compound.

Embodiment 48 is directed to the method of embodiment 47, wherein the nucleic acid is DNA.

Embodiment 49 is directed to the method of embodiment 47 or 48, the compound that interacts with nucleic acid is selected from the group consisting of PicoGreen™ dye, Hoechst 33342, 7-aminoactinomycin-D (7-AAD), and 4′6′-diamidino-2-phenylindole (DAPI).

Embodiment 50 is directed to the method of any one of embodiments 1 to 49, wherein at least one assay component selected from the group consisting of the analyte, an analyte-associating moiety, the signal donor, and the activatable compound is attached to a solid support.

Embodiment 51 is directed to method of embodiment 50, wherein the solid support comprises a polymer.

Embodiment 52 is directed to the method of embodiment 51, wherein the polymer is selected from the group consisting of nitrocellulose, cellulose acetate, poly(vinyl chloride), polyacrylamide, cross-linked dextran, agarose, polyacrylate, polyethylene, polypropylene, poly(4-methylbutene), polystyrene, polymethacrylate, poly(ethylene terephthalate), nylon, and poly(vinyl butyrate).

Embodiment 53 is directed to the method of embodiment 50, wherein the solid support comprises a synthetic latex.

Embodiment 54 is directed to the method of embodiment 53, wherein the synthetic latex is a substituted polyethylene selected from the group consisting of polystyrene-butadiene, polyacrylamide polystyrene, polystyrene with amino groups, polyacrylic acid, polymethacrylic acid, acrylonitrile-butadiene, styrene copolymers, polyvinyl acetate-acrylate, polyvinyl pyridine, and vinyl-chloride acrylate copolymers.

Embodiment 55 is directed to the method of any one of embodiments 50 to 54, wherein the solid support is in the form of a bead.

Embodiment 56 is directed to the method of any one of embodiments 1 to 55, wherein the sample is in a plate comprising a number of wells.

Embodiment 57 is directed to the method of embodiment 56, wherein the number of wells is selected from the group consisting of 6, 12, 24, 48, 96, 384, 1536, 3456, and 9600 wells.

Embodiment 58 is directed to the method of any one of embodiments 1 to 57, wherein a further assay is performed on the sample, and the assay is selected from the group consisting of bio-layer interferometry, DNA sequencing, enzyme-linked immunosorbent assay (ELISA), immunofluorescence staining, affinity chromatography, high-performance liquid chromatography (HP-LC), liquid chromatography mass spectrometry (LC-MS), size-exclusion chromatography, solid-phase extraction mass spectrometry (SPE-MS), and surface plasmon resonance.

In practicing the present invention, many conventional techniques in molecular biology, microbiology, and recombinant DNA technology are optionally used. Such conventional techniques relate to vectors, host cells, and recombinant methods. These techniques are well known and are explained in, for example, Berger and Kimmel, Guide to Molecular Cloning Techniques, Methods in Enzymology volume 152 Academic Press, San Diego, Calif.; Sambrook et al., Molecular Cloning—A Laboratory Manual (3rd Ed.), Vol. 1-3, Cold Spring Harbor Laboratory, Cold Spring Harbor, N.Y., 2000; and Current Protocols in Molecular Biology, F. M. Ausubel et al., eds., Current Protocols, a joint venture between Greene Publishing Associates, Inc. and John Wiley & Sons, Inc., (supplemented through 2006). Other useful references, for example for cell isolation and culture and for subsequent nucleic acid or protein isolation, include Freshney (1994) Culture of Animal Cells, a Manual of Basic Technique, third edition, Wiley-Liss, New York and the references cited therein; Payne et al. (1992) Plant Cell and Tissue Culture in Liquid Systems, John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York); and Atlas and Parks (Eds.) The Handbook of Microbiological Media (1993) CRC Press, Boca Raton, Fla. Methods of making nucleic acids (for example, by in vitro amplification, purification from cells, or chemical synthesis), methods for manipulating nucleic acids (for example, by site-directed mutagenesis, restriction enzyme digestion, ligation, etc.), and various vectors, cell lines, and the like useful in manipulating and making nucleic acids are described in the above references. In addition, essentially any polynucleotide (including labeled or biotinylated polynucleotides) can be custom or standard ordered from any of a variety of commercial sources.

The present invention has been described in terms of particular embodiments found or proposed to comprise certain modes for the practice of the invention. It will be appreciated by those of ordinary skill in the art that, in light of the present disclosure, numerous modifications and changes can be made in the particular embodiments exemplified without departing from the intended scope of the invention. Any embodiments or features of embodiments can be combined with one another, and such combinations are expressly encompassed within the scope of the present invention.

All cited references, including patent publications, are incorporated herein by reference in their entirety. Nucleotide and other genetic sequences, referred to by published genomic location or other description, are also expressly incorporated herein by reference.

We claim:

1. A method for determining the presence or amount of an analyte in a sample that is in the active form of the analyte, the method comprising:
(i) selecting a population of host cells having a genetic diversity, the genetic diversity comprising a plurality of genetic variants, wherein at least some of the population of host cells comprise a polynucleotide encoding the analyte;
(ii) combining a sample comprising a population of host cells comprising a single genetic variant from the selected population of host cells and wherein the population of host cells expresses the analyte, a first complex comprising a signal donor and a first analyte-associating moiety, and a second complex comprising an activatable compound and a second analyte-associating moiety;
wherein the sample comprises active analyte that is in the active form of the analyte and inactive analyte that is not in the active form of the analyte; and
wherein the active analyte associates with both the first complex and the second complex, and the inactive analyte does not associate with both the first complex and the second complex;
(iii) initiating the transfer of a signal from the signal donor in the first complex, wherein the signal is received by the activatable compound in the second complex that is associated with an active analyte associated with both the first complex and the second complex; and
(iv) detecting the output from the activatable compound.

2. A method for determining the presence or amount of an analyte in a sample, the method comprising:
(i) selecting a population of host cells having a genetic diversity, the genetic diversity comprising a plurality of genetic variants, wherein at least some of the population of host cells comprise a polynucleotide encoding the analyte;
(ii) combining a sample comprising a population of host cells comprising a single genetic variant from the selected population of host cells and wherein the population of host cells expresses the analyte with a first complex comprising a signal donor and a first analyte-associating moiety, and a second complex comprising an activatable compound and a second analyte-associating moiety;
(iii) initiating the transfer of a signal from the signal donor to the activatable compound, wherein the signal causes the activatable compound to produce detectable output; and
(iv) detecting the output from the activatable compound.

3. The method of claim 2, further comprising:
contacting the sample with a third complex comprising a second activatable compound and a third analyte-associating moiety;
wherein the transfer of the signal from the signal donor to the second activatable compound causes the second activatable compound to produce detectable output; and
detecting the output from the second activatable compound.

4. A method for determining the presence or amount of an analyte in a sample, the method comprising:
(i) selecting a population of host cells having a genetic diversity, the genetic diversity comprising a plurality of genetic variants, wherein at least some of the population of host cells comprise a polynucleotide encoding the analyte;
(ii) providing a covalently labeled analyte that comprises the analyte connected through a covalent bond to a first detection reagent selected from the group consisting of a signal donor and an activatable compound;
(ii) contacting a sample comprising a population of host cells comprising a single genetic variant from the selected population of host cells and wherein the population of host cells expresses the analyte with the covalently labeled analyte and with an analyte-associating moiety connected to a second detection reagent selected from the group consisting of a signal donor and an activatable compound, wherein the first detection reagent is not the second detection reagent;
(iii) initiating the transfer of a signal from the signal donor to the activatable compound, wherein the signal causes the activatable compound to produce detectable output; and
(iv) detecting the output from the activatable compound.

5. The method of any one of claims 2 to 4, wherein the sample comprises active analyte that is in the active form of the analyte and inactive analyte that is not in the active form of the analyte, wherein the active analyte associates with both the first complex and the second complex, and the inactive analyte does not associate with both the first complex and the second complex.

6. The method of any one of claims 1 to 4, wherein selecting the population of host cells having genetic diversity and encoding the analyte comprises:
culturing a population of host cells, whereby the analyte is expressed by a subpopulation of the host cells of the population, the subpopulation thereby comprising expressing host cells;
labeling at least some of the expressing host cells of the subpopulation, wherein the labeling comprises associating the gene product of interest with a detectable moiety, thereby producing labeled expressing host cells; and
selecting a subset of labeled expressing host cells, wherein the selecting comprises detecting the detectable moiety by a cell-sorting apparatus.

7. The method of any one of claims 1 to 4, wherein the analyte comprises a polypeptide, a protein, a glycoprotein, a phosphoprotein, a proteolipid, an antibody, a polypeptide comprising one or more domains or fragments of any of the preceding, and a nucleic acid.

8. The method of any one of claims 1 to 4, wherein the analyte is a homomultimer and/or the analyte has properly formed disulfide bonds.

9. The method of any one of claims 1 to 4, wherein the analyte is a homomultimer and the analyte-associating moiety of the first complex is the same as the analyte-associating moiety of the second complex.

10. The method of any one of claims 1 to 4, further comprising contacting an assay component with a first antibody that specifically binds the assay component, where the assay component is selected from the group consisting of the analyte and the analyte-associating moiety.

11. The method of claim 10, further comprising contacting the first antibody with a second antibody that specifically binds the first antibody.

12. The method of any one of claims 1 to 4, wherein the analyte-associating moiety is a multimer that can interact with the analyte at multiple sites on the analyte.

13. The method of any one of claims 1 to 4, wherein the analyte-associating moiety is attached to a detection reagent selected from the group consisting of a signal donor and an activatable complex.

14. The method of claim 13, wherein the analyte-associating moiety is attached to the detection reagent through a connector.

15. The method of any one of claims 1 to 4, wherein the signal donor is activated by an enzyme and/or the signal donor is activated by irradiation.

16. The method of any one of claims 1 to 4, wherein the signal donor produces a fluorescence resonance transfer signal, and/or the signal donor produces a chemical signal.

17. The method of claim 16, wherein the chemical signal is a reactive oxygen species.

18. The method of any one of claims 1 to 4, wherein the signal donor is a sensitizer.

19. The method of claim 18, wherein the sensitizer is a haloperoxidase or a photosensitizer.

20. The method of any one of claims 1 to 4, wherein the activatable compound is a photoactivable compound.

21. The method of claim 20, wherein the photoactivable compound emits light by fluorescence.

22. The method of claim 20, wherein the photoactivable compound undergoes a chemical reaction with singlet oxygen.

23. The method of any one of claims 1 to 4, further comprising measuring the optical density of the sample.

24. The method of any one of claims 1 to 4, further comprising adding a compound that interacts with nucleic acid to the sample, irradiating the sample to excite the compound that interacts with nucleic acid, and measuring the light emitted by the excited compound.

25. The method of any one of claims 1 to 4, wherein at least one assay component selected from the group consisting of the analyte, an analyte-associating moiety, the signal donor, and the activatable compound is attached to a solid support.

26. The method of claim 25, wherein the solid support is in the form of a bead.

27. The method of any one of claims 1 to 4, wherein a further assay is performed on the sample, and the assay is selected from the group consisting of bio-layer interferometry, DNA sequencing, enzyme-linked immunosorbent assay (ELISA), immunofluorescence staining, affinity chromatography, high-performance liquid chromatography (HP-LC), liquid chromatography mass spectrometry (LC-MS), size-exclusion chromatography, solid-phase extraction mass spectrometry (SPE-MS), and surface plasmon resonance.


SEQUENCE LISTING (3 sequences)

SEQ ID NO: 1 - protein, 451 aa, Artificial Sequence
  Trastuzumab heavy chain
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Artificial Sequence
  Trastuzumab light chain
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 15 aa, Artificial Sequence
  AviTag(TM) peptide for covalent attachment of biotin
GLNDIFEAQKIEWHE